(19) United States
(12) Patent Application Publication
(10) Pub. No.: US 2008/0262005 A1
(43) Pub. Date: Oct. 23, 2008

(54) Uracil-Type Gonadotropin-Releasing Hormone Receptor Antagonists and Methods Related Thereto

(75) Inventors: Yun-Fei Zhu, San Diego, CA (US); Zhiqiang Gou, San Diego, CA (US); Mi Chen, San Diego, CA (US); Liren Zhao, San Diego, CA (US)

Correspondence Address: SEED INTELLECTUAL PROPERTY LAW GROUP PLLC, 701 FIFTH AVE, SUITE 5400, SEATTLE, WA, 98104, US

(73) Assignee: NEUROCRINE BIOSCIENCES, INC., San Diego, CA (US)

(21) Appl. No.: 11/568,961

(22) PCT Filed: May 13, 2005
(86) PCT No.: PCT/US05/16906

§ 371 Date: Sep. 10, 2007

Related U.S. Application Data
(60) Provisional application No. 60/571,207, filed on May 14, 2004.

(51) Int. Cl. A61K 31/513; C07D 239/54; A61P 5/00; A61P 43/00; A61P 15/18

(52) U.S. Cl. 514/274; 544/313; 544/311; 544/309

(57) ABSTRACT

Compounds having utility as GnRH receptor antagonists and for treatment of a variety of sex-hormone related conditions in both men and women. Such compounds have the following structure (I): (I) wherein R₁ₐ, R_{1b}, R_{1c}, R₂ₐ, R_{2b}, R₃, R₄, R₅, R₆, R₇, n and X are as defined herein, including stereoisomers, prodrugs and pharmaceutically acceptable salts thereof. Also disclosed are compositions containing a compound of structure (I) in combination with a pharmaceutically acceptable carrier, as well as methods relating to the use thereof for antagonizing gonadotropin-releasing hormone in a subject in need thereof.

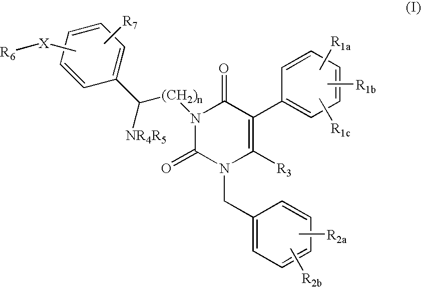

### BACKGROUND OF THE INVENTION

1. Field of the Invention

This invention relates generally to gonadotropin-releasing hormone (GnRH) receptor antagonists, and to methods of treating disorders by administration of such antagonists to a warm-blooded animal in need thereof.

2. Description of the Related Art

Gonadotropin-releasing hormone (GnRH), also known as luteinizing hormone-releasing hormone (LHRH), is a decapeptide (pGlu-His-Trp-Ser-Tyr-Gly-Leu-Arg-Pro-Gly-NH2) that plays an important role in human reproduction. GnRH is released from the hypothalamus and acts on the pituitary gland to stimulate the biosynthesis and release of luteinizing hormone (LH) and follicle-stimulating hormone (FSH). LH released from the pituitary gland is responsible for the regulation of gonadal steroid production in both males and females, while FSH regulates spermatogenesis in males and follicular development in females.

Due to its biological importance, synthetic antagonists and agonists to GnRH have been the focus of considerable attention, particularly in the context of prostate cancer, breast cancer, endometriosis, uterine leiomyoma, and precocious puberty. For example, peptidic GnRH agonists, such as leuprorelin (pGlu-His-Trp-Ser-Tyr-d-Leu-Leu-Arg-Pro-NHEt), have been used to treat such conditions. Such agonists appear to function by binding to the GnRH receptor in the pituitary gonadotropins, thereby inducing the synthesis and release of gonadotropins. Chronic administration of GnRH agonists depletes gonadotropins and subsequently down-regulates the receptor, resulting in suppression of steroidal hormones after some period of time (e.g., on the order of 2-3 weeks following initiation of chronic administration).

In contrast, GnRH antagonists are believed to suppress gonadotropins from the onset, and thus have received the most attention over the past two decades. To date, some of the primary obstacles to the clinical use of such antagonists have been their relatively low bioavailability and adverse side effects caused by histamine release. However, several peptidic antagonists with low histamine release properties have been reported, although they still must be delivered via sustained delivery routes (such as subcutaneous injection or intranasal spray) due to limited bioavailability.

In view of the limitations associated with peptidic GnRH antagonists, a number of nonpeptidic compounds have been proposed. For example, Cho et al. (J. Med. Chem. 41:4190-4195, 1998) discloses thieno[2,3-b]pyridin-4-ones for use as GnRH receptor antagonists; U.S. Pat. Nos. 5,780,437 and 5,849,764 teach substituted indoles as GnRH receptor antagonists (as do published PCTs WO 97/21704, 98/55479, 98/55470, 98/55116, 98/55119, 97/21707, 97/21703 and 97/21435); published PCT WO 96/38438 discloses tricyclic diazepines as GnRH receptor antagonists; published PCTs W097/14682, 97/14697 and 99/09033 disclose quinoline and thienopyridine derivatives as GnRH antagonists; published PCTs WO 97/44037, 97/44041, 97/44321 and 97/44339 teach substituted quinolin-2-ones as GnRH receptor antagonists; and published PCT WO 99/33831 discloses certain phenyl-substituted fused nitrogen-containing bicyclic compounds as GnRH receptor antagonists. Published PCT WO 01/55119 discloses substituted uracils useful as GnRH antagonists, while published PCTs WO 02/066459 and WO 02/11732 disclose the use of indole derivatives and novel bicyclic and tricyclic pyrrolidine derivatives as GnRH antagonists, respectively. Other published PCTs which disclose compounds and their use as GnRH antagonists include WO 00/69859, WO 01/29044, WO 03/013528, WO 03/011870, WO 03/011841, WO 03/011839, WO 03/011293, WO05/007164, W005/007165 and W005/007633.

While significant strides have been made in this field, there remains a need in the art for effective small molecule GnRH receptor antagonists. There is also a need for pharmaceutical compositions containing such GnRH receptor antagonists, as well as methods relating to the use thereof to treat, for example, sex-hormone related conditions. The present invention fulfills these needs, and provides other related advantages.

### BRIEF SUMMARY OF THE INVENTION

In brief, this invention is generally directed to compounds that have activity as gonadotropin-releasing hormone (GnRH) receptor antagonists, as well as to methods for their preparation and use, and to pharmaceutical compositions containing the same. More specifically, the compounds of this invention have the following general structure (I):

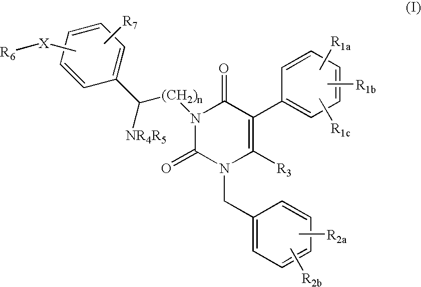

including stereoisomers, prodrugs and pharmaceutically acceptable salts thereof, wherein R₁ₐ, R_{1b}, R_{1c}, R₂ₐ, R_{2b}, R₃, R₄, R₅, R₆, R₇, n, and X are as defined below.

The compounds of this invention have utility over a wide range of therapeutic applications, and may be used to treat a variety of sex-hormone related conditions in both men and women, as well as a mammal in general (also referred to herein as a “subject”). For example, such conditions include endometriosis, uterine fibroids, polycystic ovarian disease, hirsutism, precocious puberty, gonadal steroid-dependent neoplasia such as cancers of the prostate, breast and ovary, gonadotrophe pituitary adenomas, sleep apnea, irritable bowel syndrome, premenstrual syndrome, benign prostatic hypertrophy, contraception and infertility (e.g., assisted reproductive therapy such as in vitro fertilization). The compounds of this invention may also be useful as an adjunct to treatment of growth hormone deficiency and short stature, and for the treatment of systemic lupus erythematosis. The compounds may also be useful in combination with androgens, estrogens, progesterones, and antiestrogens and antiprogestogens for the treatment of endometriosis, fibroids, and in contraception, as well as in combination with an angiotensin-converting enzyme inhibitor, an angiotensin II-receptor antagonist, or a renin inhibitor for the treatment of uterine fibroids. In addition, the compounds may be used in combination with bisphosphonates and other agents for the treatment and/or prevention of disturbances of calcium, phosphate and bone metabolism, and in combination with estrogens, progesterones and/or androgens for the prevention or treatment of bone loss or hypogonadal symptoms such as hot flashes during therapy with a GnRH antagonist.

The compounds of the present invention, in addition to their GnRH receptor antagonist activity, possess a reduced interaction with the major metabolic enzymes in the liver, namely the Cytochrome P450 enzymes. This family of enzymes, which includes the subtypes CYP2D6 and CYP3A4, is responsible for the metabolism of drugs and toxins leading to their disposition from the body. Inhibition of these enzymes can lead to life-threatening conditions where the enzyme is not able to perform this function.

The methods of this invention include administering an effective amount of a compound of structure (I) above, preferably in the form of a pharmaceutical composition, to a mammal in need thereof. Thus, in still a further embodiment, pharmaceutical compositions are disclosed containing one or more compounds of this invention in combination with a pharmaceutically acceptable carrier and/or diluent.

These and other aspects of the invention will be apparent upon reference to the following detailed description. To this end, various references are set forth herein which describe in more detail certain background information, procedures, compounds and/or compositions, and are each hereby incorporated by reference in their entirety.

### DETAILED DESCRIPTION OF THE INVENTION

As mentioned above, the present invention is directed generally to compounds generally having activity as gonadotropin-releasing hormone (GnRH) receptor antagonists. The compounds of this invention have the following structure (I):

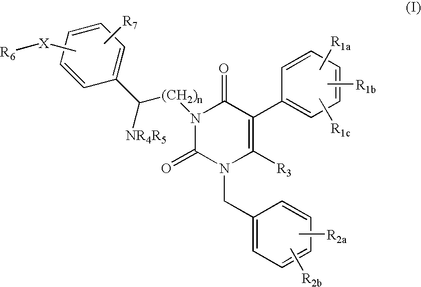

and stereoisomers, prodrugs and pharmaceutically acceptable salts, esters and solvates thereof,

wherein:
R₁ₐ, R_{1b} and R_{1c} are the same or different and independently hydrogen, halogen, C₁₋₄alkyl or alkoxy;
R₂ₐ and R_{2b} are the same or different and independently hydrogen, halogen, trifluoromethyl, cyano or —SO₂CH₃;
R₃ is hydrogen or methyl;
R₄ and R₅ are the same or different and independently hydrogen or lower alkyl;
R₆ is —COOH or an acid isostere;
R₇ is hydrogen, halogen or C₁₋₆alkyl;
n is 1 or 2; and
X is —(C₁₋₆alkanediyl)-O— (with the R₆ moiety being joined to the C₁₋₆ alkanediyl moiety, as opposed to the oxygen atom), where C₁₋₆alkanediyl is optionally substituted with from 1 to 3 C₁₋₄alkyl groups.

As used herein, the above terms have the following meaning:

“C₁₋₆alkyl” means a straight chain or branched, noncyclic or cyclic, unsaturated or saturated aliphatic hydrocarbon containing from 1 to 6 carbon atoms. Representative saturated straight chain C₁₋₆alkyl include methyl, ethyl, n-propyl, n-butyl, n-pentyl, n-hexyl, and the like; while saturated branched alkyls include isopropyl, sec-butyl, isobutyl, tert-butyl, isopentyl, and the like. Representative saturated cyclic alkyls include cyclopropyl, cyclobutyl, cyclopentyl, cyclohexyl, and the like; while unsaturated cyclic alkyls include cyclopentenyl and cyclohexenyl, and the like. Unsaturated alkyls contain at least one double or triple bond between adjacent carbon atoms (referred to as an “alkenyl” or “alkynyl”, respectively). Representative straight chain and branched alkenyls include ethylenyl, propylenyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, and the like; while representative straight chain and branched alkynyls include acetylenyl, propynyl, 1-butynyl, 2- butynyl, 1-pentynyl, 2-pentynyl, 3-methyl-1-butynyl, and the like.

“C₁₋₄alkyl” as the same meaning as given above for C₁₋₆alkyl, but containing from 1 to 4 carbon atoms (as opposed to 1 to 6 carbon atoms).

“C₁₋₆alkanediyl” means a divalent C₁₋₆alkyl from which two hydrogen atoms are taken from the same carbon atom or from different carbon atoms, such as —CH₂—, —CH₂CH₂—, —CH₂CH₂CH₂—, —CH(CH₃)CH₂CH₂—, —CH₂C(CH₃)₂CH₂—, and the like.

“Halogen” means fluoro, chloro, bromo or iodo, typically fluoro and chloro.

“Hydroxy” means —OH.

“Alkoxy” means —O—(C₁₋₆alkyl).

“Cyano” means —CN.

“Acid isostere” means a moiety that exhibits properties similar to carboxylic acid and, more specifically, a moiety having a pKa of less than 8 and preferably less than 7. Representative acid isosteres include tetrazole, 3H-[1,3,4]oxadiazol-2-one, [1,2,4]oxadiazol-3-one, 1,2-dihydro-[1,2,4]triazol-3-one, 2H-[1,2,4]oxadiazol-5-one, triazole substituted with a sulfonyl or sulfoxide group, imidazole substituted with a sulfonyl or sulfoxide group, [1,2,4]-oxadiazolidine-3,5-dione, [1,2,4]-thiadiazolidine-3,5-dione, imidazolidine-2,4-dione, imidazolidine-2,4,5-trione, pyrrolidine-2,5-dione and pyrrolidine-2,3,5-trione. Acid isosteres also include —C(═O)NHSO₂NRₐR_{b}, —C(═O)NHSO₂R_{b}, —C(═O)NHC(═O)NRₐR_{b} and —C(═O)NHC(═O)R_{b}, where Rₐ is hydrogen or C₁₋₄alkyl and R_{b} is C₁₋₄alkyl.

In one embodiment of the invention, R₄ and R₅ are both hydrogen, and compounds of this invention have the following structure (II):

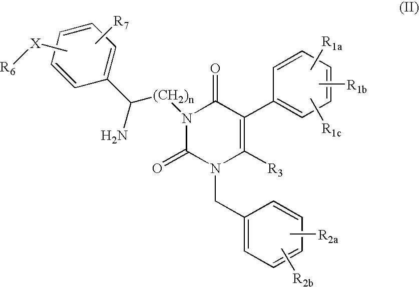

In another embodiment, n is 1 or 2, and compounds of this invention have one of the following structures (III) or (IV), respectively.

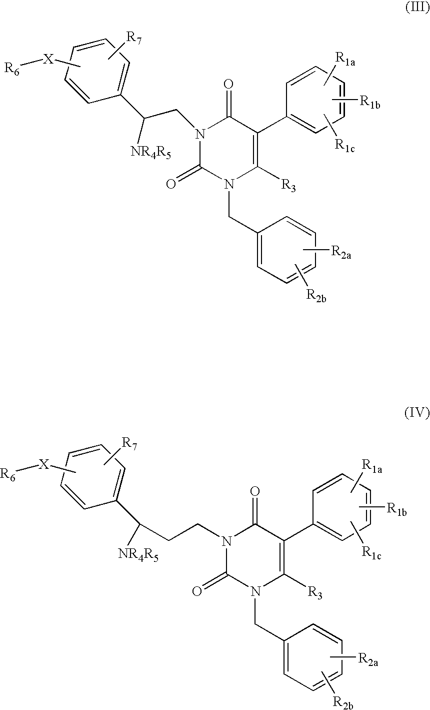

In another embodiment, the “—X—R₆” moiety is ortho, meta or para to the point of attachment of the phenyl ring, n is 1, and compounds of this invention have one of the following structures (V), (VI) or (VII), respectively:

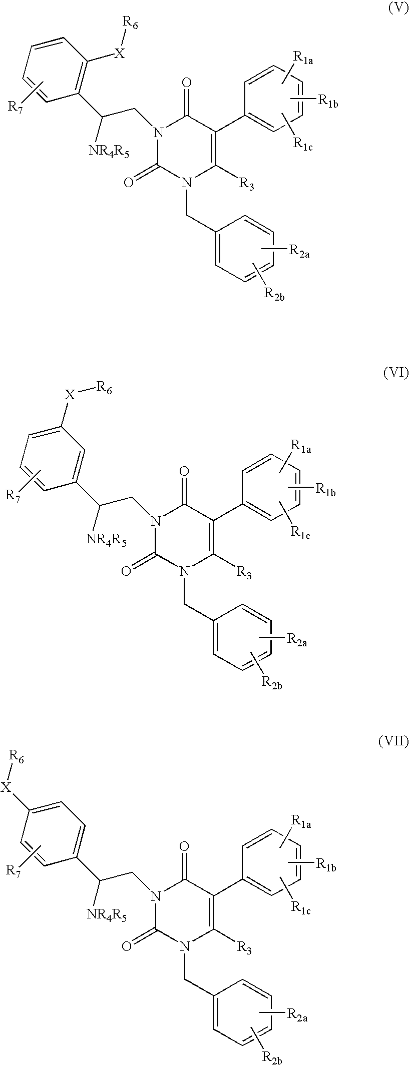

In another embodiment, R₁ₐ, R_{1b} and R_{1c} are independently hydrogen, halogen or alkoxy; and, in a more specific embodiment, R_{1c} is hydrogen, and R₁ₐ and R_{1b} are independently hydrogen, halogen (e.g., fluoro or chloro) or alkoxy (e.g., methoxy or ethoxy).

In other embodiments, R₂ₐ and R_{2b} are independently hydrogen, trifluoromethyl, halogen (e.g., fluoro or chloro) or —SO₂CH₃.

In still further embodiments, representative X moieties include —CH₂—O—, —CH₂CH₂—O—, —CH₂CH₂CH₂—O—, and —CH₂CH₂CH₂CH₂—O—.

The compounds of the present invention may be prepared by known organic synthesis techniques, including the methods described in more detail in the Examples. In general, the compounds of structure (I) above may be made by the following Reaction Schemes 1 through 5, wherein all substituents are as defined above unless indicated otherwise.

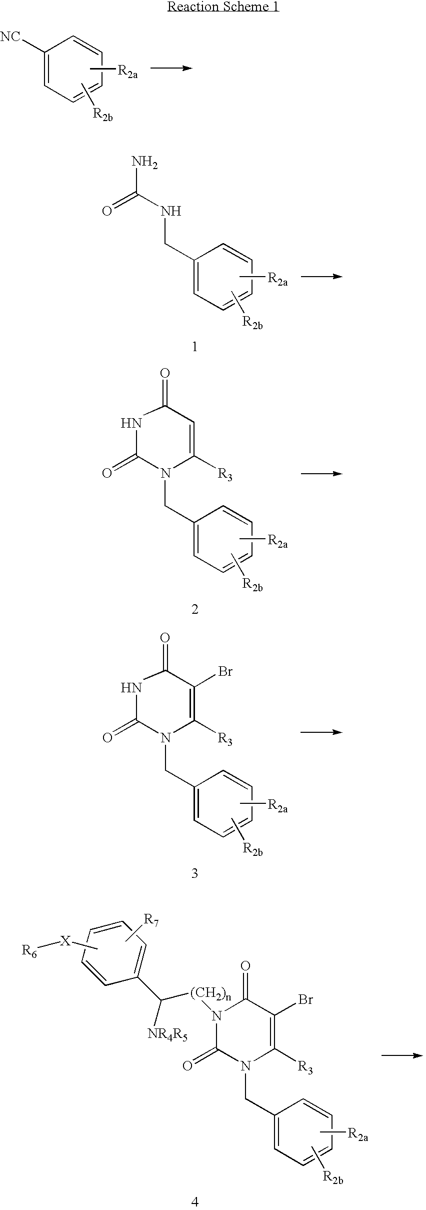

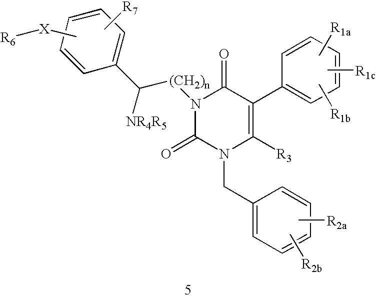

An appropriately substituted benzonitrile may be reduced using an appropriate reagent such as borane in THF and then forms urea 1. Cyclization with a reagent such as diketene gives compound 2 which may be brominated with bromine in acetic acid, N-bromosuccinimide or other brominating agent to give compound 3. Alkylation via Mitsunobu or other conditions or reductive amination gives compound 4 and Suzuki condensation with a boronic acid or boronic acid ester gives compound 5. It is possible to alter the order of the various reductive amination, alkylation, bromination and Suzuki condensation steps to give compounds of the present invention.

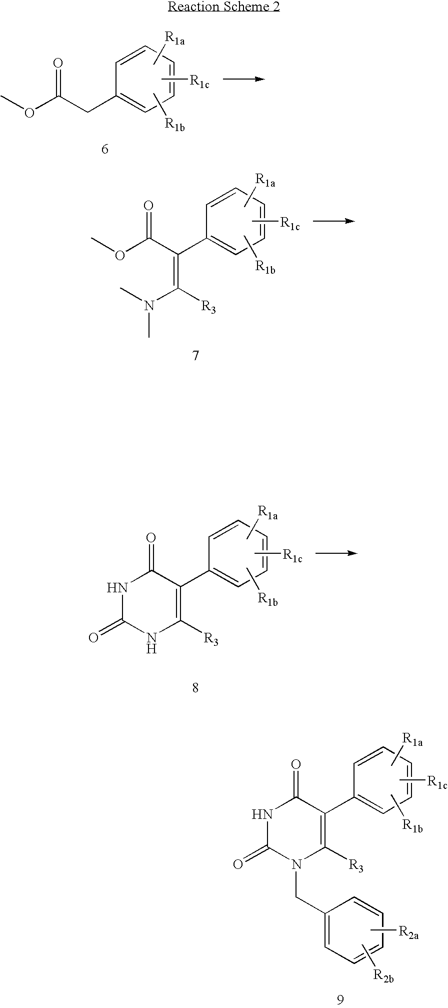

Substituted phenylacetic acid ester 6 (made from the corresponding acid or purchased) and reagent such as dimethylformamide dimethylacetal are condensed to give 7. Cyclization with urea gives a compound of formula 8. Alkylation using, for example, a substituted benzyl bromide gives 9 which may be alkylated with an appropriate alkyl halide or undergo a Mitsunobu reaction with an appropriate alcohol to give 5.

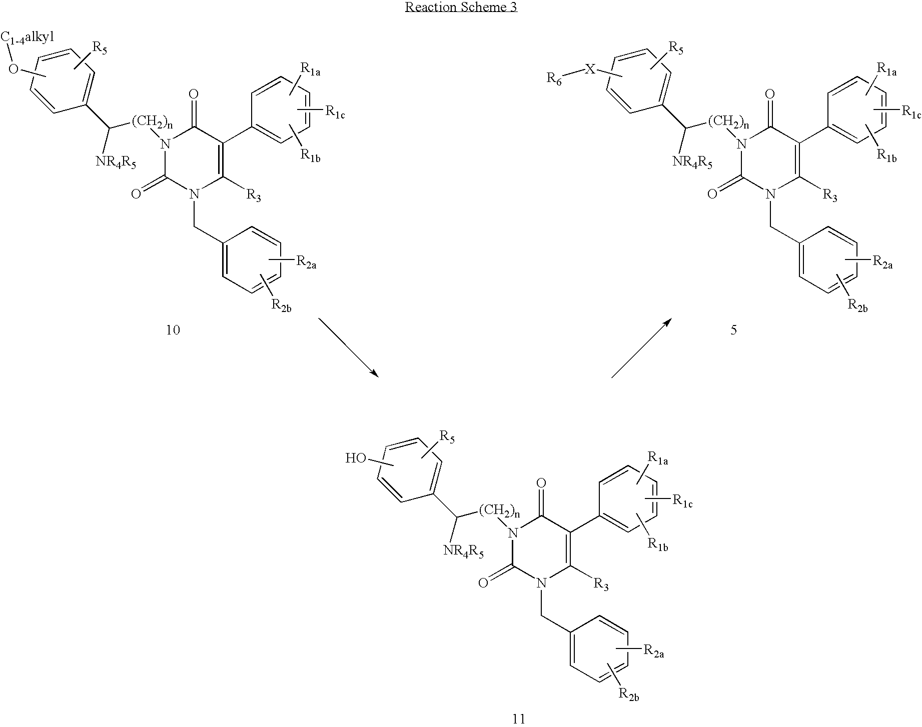

Compound 10 may be dealkylated with an appropriate acid such as HBr or BBr₃ to give compound 11. Alkylation with an appropriate acid or acid isostere containing group such as an alkyl halide which contains an acid or acid isostere functionality gives compound 5.

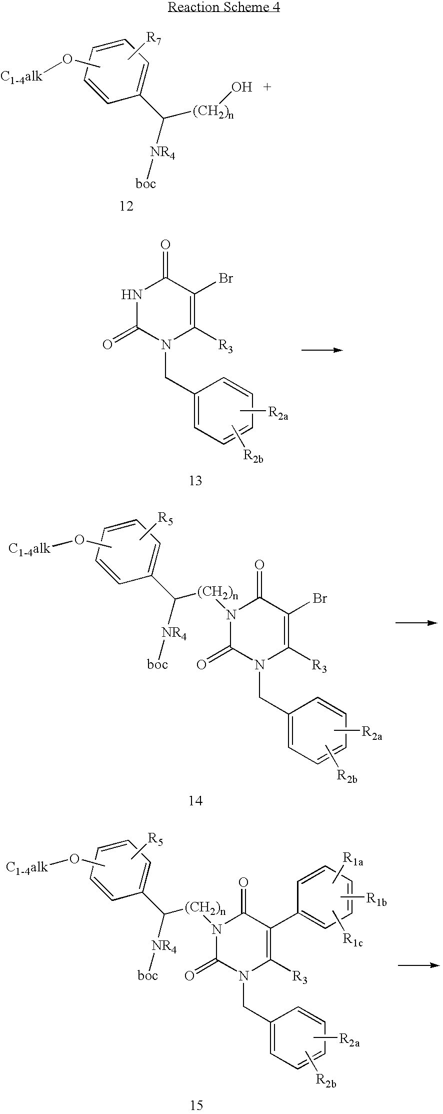

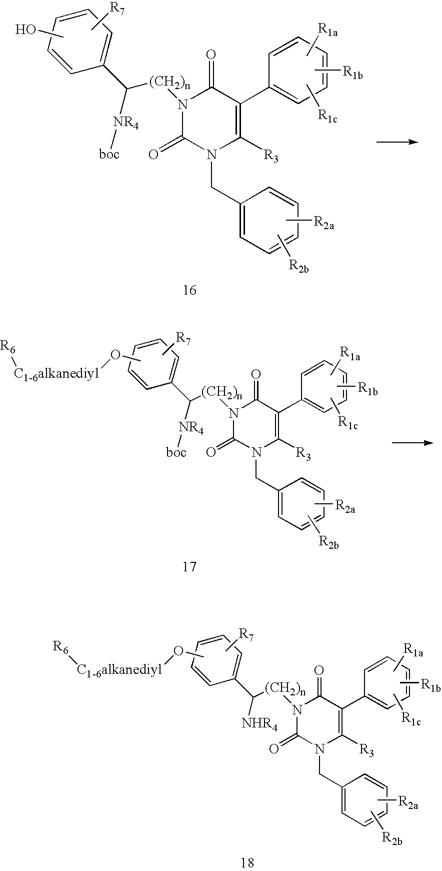

Compounds 12 and 13 are condensed in a procedure such as a Mitsunobu reaction using reagents such as triphenylphosphine and diisopropylazodicarboxylate in a solvent such as tetrahydrofuran to give compound 14. A Suzuki type coupling using a palladium catalyst and a boronic acid or boronic ester gives compound 15. Dealkylation using an acid such as boron tribromide in methylene chloride gives the alcohol and deprotected amine. The amine may be protected with an appropriate protecting group such as by reaction with di-tert-butyldicarbonate to give the BOC protected 16. Alkylation with an appropriately substituted acid, ester or acid isostere containing alkyl halide gives compound 17 which may be deprotected to give 18.

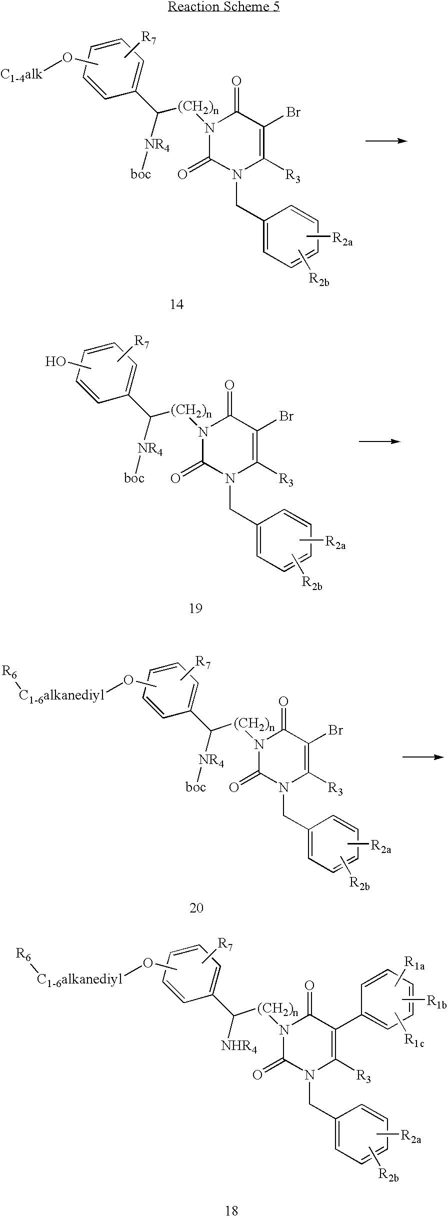

By varying the reaction order of the earlier schemes, compound 14 is dealkylated to give alcohol 19. Alkylation with an appropriate alkyl halide which is substituted with an acid, an acid isostere, or a group which acts as a precursor for an acid or acid isostere such as an ester gives compound 20. Suzuki coupling with boronic acid or boronic ester followed by deprotection of ester functionalities or protecting groups such as a BOC group gives 18.

The compounds of the present invention may generally be utilized as the free acid or free base. Alternatively, the compounds of this invention may be used in the form of acid or base addition salts. Acid addition salts of the free amino compounds of the present invention may be prepared by methods well known in the art, and may be formed from organic and inorganic acids. Suitable organic acids include maleic, fumaric, benzoic, ascorbic, succinic, methanesulfonic, acetic, trifluoroacetic, oxalic, propionic, tartaric, salicylic, citric, gluconic, lactic, mandelic, cinnamic, aspartic, stearic, palmitic, glycolic, glutamic, and benzenesulfonic acids. Suitable inorganic acids include hydrochloric, hydrobromic, sulfuric, phosphoric, and nitric acids. Base addition salts included those salts that form with the carboxylate anion and include salts formed with organic and inorganic cations such as those chosen from the alkali and alkaline earth metals (for example, lithium, sodium, potassium, magnesium, barium and calcium), as well as the ammonium ion and substituted derivatives thereof (for example, dibenzylammonium, benzylammonium, 2-hydroxyethylammonium, and the like). Thus, the term “pharmaceutically acceptable salt” of structure (I) is intended to encompass any and all acceptable salt forms.

In addition, prodrugs are also included within the context of this invention. Prodrugs are any covalently bonded carriers that release a compound of structure (I) in vivo when such prodrug is administered to a patient. Prodrugs are generally prepared by modifying functional groups in a way such that the modification is cleaved, either by routine manipulation or in vivo, yielding the parent compound. Prodrugs include, for example, compounds of this invention wherein hydroxy, amine or sulfhydryl groups are bonded to any group that, when administered to a patient, cleaves to form the hydroxy, amine or sulflhydryl groups. Thus, representative examples of prodrugs include (but are not limited to) acetate, formate and benzoate derivatives of alcohol and amine functional groups of the compounds of structure (I). Further, in the case of a carboxylic acid (—COOH), esters may be employed, such as methyl esters, ethyl esters, and the like.

With regard to stereoisomers, the compounds of structure (I) may have chiral centers and may occur as racemates, racemic mixtures and as individual enantiomers or diastereomers. All such isomeric forms are included within the present invention, including mixtures thereof. Furthermore, some of the crystalline forms of the compounds of structure (I) may exist as polymorphs, which are included in the present invention. In addition, some of the compounds of structure (I) may also form solvates with water or other organic solvents. Such solvates are similarly included within the scope of this invention.

The effectiveness of a compound as a GnRH receptor antagonist may be determined by various assay techniques. Assay techniques well known in the field include the use of cultured pituitary cells for measuring GnRH activity (Vale et al., Endocrinology 91:562-572, 1972) and the measurement of radioligand binding to rat pituitary membranes (Perrin et al., Mol. Pharmacol. 23:44-51, 1983) or to membranes from cells expressing cloned receptors as described below. Other assay techniques include (but are not limited to) measurement of the effects of GnRH receptor antagonists on the inhibition of GnRH-stimulated calcium flux, modulation of phosphoinositol hydrolysis, and the circulating concentrations of gonadotropins in the castrate animal. Descriptions of these techniques, the synthesis of radiolabeled ligand, the employment of radiolabeled ligand in radioimmunoassay, and the measurement of the effectiveness of a compound as a GnRH receptor antagonist follow.

Inhibition of GnRH Stimulated LH Release

Suitable GnRH antagonists are capable of inhibiting the specific binding of GnRH to its receptor and antagonizing activities associated with GnRH. For example, inhibition of GnRH stimulated LH release in immature rats may be measured according to the method of Vilchez-Martinez (Endocrinology 96:1130-1134, 1975). Briefly, twenty-five day old male Spraque-Dawley rats are administered an GnRH antagonist in saline or other suitable formulation by oral gavage, subcutaneous injection, or intravenous injection. This is followed by subcutaneous injection of 200 ng GnRH in 0.2 ml saline. Thirty minutes after the last injection, the animals are decapitated and trunk blood is collected. After centrifugation, the separated plasma is stored at —20 ° C. until determination of the concentrations of LH and/or FSH by radioimmunoassay (see below.)

Rat Anterior Pituitary Cell Culture Assay of GnRH Antagonists

Anterior pituitary glands are collected from 7-week-old female Sprague-Dawley rats and the harvested glands are digested with collagenase in a dispersion flask for 1.5 hr at 37° C. After collagenase digestion, the glands are further digested with neuraminidase for 9 min at 37 ° C. The digested tissue is then washed with 0.1% BSA/McCoy's 5A medium, and the washed cells are suspended in 3% FBS/0.1 BSA/McCoy's 5A medium and plated onto 96-well tissue culture plates at a cell density of 40,000 cells per well in 200 μl medium. The cells are then incubated at 37° C. for 3 days. For assay of an GnRH antagonist, the incubated cells are first washed with 0.1% BSA/McCoy's 5A medium once, followed by addition of the test sample plus 1 nM GnRH in 200 μl 0.1% BSA/McCoy's 5A medium in triplicate wells. Each sample is assayed at 5-dose levels to generate a dose-response curve for determination of the potency on the inhibition of GnRH stimulated LH and/or FSH release. After 4-hr incubation at 37° C., the medium is harvested and the level of LH and/or FSH secreted into the medium is determined by RIA.

Membrane Binding Assays 1

Cells stably, or transiently, transfected with GnRH receptor expression vectors are harvested, resuspended in 5% sucrose and homogenized using a polytron homogenizer (2×15 sec). Nucleii are removed by centrifugation (3000×g for 5 min.), and the supernatant is centrifuged (20,000×g for 30 min, 4° C.) to collect the membrane fraction. The final membrane preparation is resuspended in binding buffer (10 mM Hepes (pH 7.5), 150 mM NaCl, and 0.1% BSA) and stored at −70° C. Binding reactions are performed in a Millipore MultiScreen 96-well filtration plate assembly with polyethylenimine coated GF/C membranes. The reaction is initiated by adding membranes (40 μg protein in 130 μl binding buffer) to 50 μl of [¹²⁵I]-labeled GnRH peptide (˜100,000 cpm) and 20 μl of competitor at varying concentrations. The reaction is terminated after 90 minutes by application of vacuum and washing (2×) with phosphate buffered saline. Bound radioactivity is measured using 96-well scintillation counting (Packard Topcount) or by removing the filters from the plate and direct gamma counting. Kᵢ values are calculated from competition binding data using non-linear least squares regression using the Prism software package (GraphPad Software).

Membrane Binding Assays 2

For additional membrane binding assays, stably transfected HEK293 cells are harvested by striking tissue culture flasks against a firm surface and collected by centrifugation at 1000×g for 5 minutes. Cell pellets are resuspended in 5% sucrose and homogenized using a polytron homogenizer for two 15 second homogenization steps. Cell homogenates are then centrifuged for 5 minutes at 3000×g to remove nuclei, and the supernatant is subsequently centrifuged for 30 minutes at 44,000×g to collect the membrane fraction. The membrane pellet is resuspended in GnRH binding buffer (10 mM HEPES, pH 7.5, 150 mM NaCl and 0.1% BSA,) and aliquots are immediately snap-frozen in liquid nitrogen and stored at —80° C. Protein content of the membrane suspension is determined using the Bio-Rad protein assay kit (Bio-Rad, Hercules, Calif.).

Competitive radioligand binding assays with membrane preparations are performed in Millipore 96-well filtration plates with GF/C membrane filters which are pre-coated with 200 μl of 0.1% polyethylenimine (Sigma, St. Louis. Mo.). Prior to use, the plates are washed 3× with phosphate buffered saline solution. Membrane fraction in GnRH binding buffer (130 μl containing 25 μg protein for human and macaque receptors or 12 μg for rat receptors) are added to wells together with 20 μl of competing ligand at varying concentrations. The binding reaction is initiated by addition of radioligand (0.1 nM in 50 μl GnRH binding buffer.) The reaction is allowed to proceed for 90 min on a platform shaker at room temperature and then terminated by placing assay plate on a Millipore vacuum manifold (Millipore, Bedford, Mass.), aspirating the solvent, and washing wells twice with 200 μl ice cold phosphate buffered saline (PBS). Filters in the wells are removed and counted in a gamma counter. Kᵢ values are calculated from each competition binding curves using non-linear least square regression and corrected for radioligand concentration using the Cheng-Prusoff equation (Prism, GraphPad Software, San Diego, Calif.) assuming a radioligand affinity of 0.5 nM. Mean Kᵢ values are calculated from the antilog of the mean of the pKᵢ values for each receptor ligand pair.

Membrane Binding Assays 3

Stably transfected human GNRH receptor RBL cells are grown to confluence. The medium is removed and the cell monolayer is washed once with DPBS. A solution of 0.5 mM EDTA/PBS (Ca⁺⁺ Mg⁺⁺ free) is added to the plate which is then incubated at 37° C. for 10 min. Cells are dislodged by gentle rapping of the flasks. The cells are collected and pelleted by centrifugation at 800 g for 10 min at 4° C. The cell pellet is then resuspended in buffer [DPBS (1.5 mM KH₂PO₄, 8.1 mM Na₂HPO₄, 2.7 mM KCl, and 138 mM NaCl) supplemented with 10 mM MgCl₂, 2 mM EGTA, pH=7.4 with NaOH]. Cell lysis is then performed using a pressure cell and applying N₂ at a pressure of 900 psi for 30 min at 4° C. Unbroken cells and larger debris are removed by centrifugation at 1200 g for 10 min at 4° C. The cell membrane supernatant is then centrifuged at 45,000 g and the resulting membrane pellet is resuspended in assay buffer and homogenized on ice using a tissue homogenizer. Protein concentrations are determined using the Coomassie Plus Protein Reagent kit (Pierce, Rockford, Ill.) using bovine serum albumin as a standard. The pellets are aliquoted and stored at −80° C. until use. Titration analysis using a range of protein concentrations determined the optimal protein concentration to be 15 μg per well final concentration.

UniFilter GF/C filter plates (Perkin Elmer, Boston Mass. ) are pretreated with a solution of 0.5% polyethyleneimine in distilled water for 30 minutes. Filters are pre-rinsed with 200 μl per well of PBS, 1% BSA (Fraction V) and 0.01% Tween-20, pH 7.4) using a cell harvester (UniFilter-96 Filtermate; Packard). Membranes are harvested by rapid vacuum filtration and washed 3 times with 250 μl of ice-cold buffer (PBS, 0.01% Tween-20, pH =7.4). Plates are air dried, 50 μl scintillation fluid (Microscint 20; Packard) is added, and the plate is monitored for radioactivity using a TopCount NXT (Packard Instruments, Ill.).

Binding experiments are performed in buffer containing 10 mM HEPES, 150 mM NaCl, and 0.1% BSA, pH =7.5. Membranes are incubated with 50 μl [¹²⁵I] His5, D-Tyr⁶ GnRH (0.2 nM final concentration) and 50 μl of small molecule competitors at concentrations ranging from 30 pM to 10 μM for a total volume in each well of 200 μl. Incubations are carried out for 2 hrs at room temperature. The reaction is terminated by rapid filtration over GF/C filters as previously described. Curve fitting is performed using Excel Fit Software (IDBS, Emeryville, Calif. ). The Ki values are calculated using the method of Cheng and Prusoff (Cheng and Prusoff, 1973) using a Kd value of 0.7 nM for the radioligand which was previously determined in saturation binding experiments.

Ca⁺⁺ Flux Measurement

To determine the inhibition of GnRH-stimulated calcium flux in cells expressing the human GnRH receptor, a 96-well plate is seeded with RBL cells stably transfected with the human GnRH receptor at a density of 50,000 cells/well and allowed to attach overnight. Cells are loaded for 1 hr at 37° C. in the following medium: DMEM with 20 mM HEPES, 10% FBS, 2 μM Fluo-4, 0.02% pluronic acid and 2.5 mM probenecid. Cells are washed 4 times with wash buffer (Hanks balanced salt, 20 mM HEPES, 2. 5 mM probenecid) after loading, leaving 150 μl in the well after the last wash. GnRH is diluted in 0.1% BSA containing FLIPR buffer (Hanks balanced salt, 20 mM HEPES) to a concentration of 20 nM and dispensed into a 96-well plate (Low protein binding). Various concentrations of antagonists are prepared in 0.1% BSA/FLIPR buffer in a third 96-well plate. Measurement of fluorescence due to GnRH stimulated (50 μl of 20 nM, or 4 nM final) Ca⁺⁺ flux is performed according to manufacturer's instructions on a FLIPR system (Molecular Devices, FLIPR 384 system, Sunnyvale, Calif.) following a 1-minute incubation with 50 μl of antagonist at varying concentrations.

Phosphoinositol Hydrolysis Assay

The procedure is modified from published protocols (W. Zhou et al; J. Biol. Chem. 270(32), ppl8853-18857, 1995). Briefly, RBL cells stably transfected with human GnRH receptors are seeded in 24 well plates at a density of 200, 000 cell/well for 24 hrs. Cells are washed once with inositol-free medium containing 10% dialyzed FBS and then labeled with luCi/mL of [myo-³H]-inositol. After 20-24 hrs, cells are washed with buffer (140 nM NaCl, 4 mM KCl, 20 mM Hepes, 8.3 mM glucose, 1 mM MgCl₂, 1 mM CaCl₂ and 0.1%BSA) and treated with native GnRH peptide in the same buffer with or without various concentrations of antagonist and 10 mM LiCl for 1 hour at 37° C. Cells are extracted with 10 mM formic acid at 4 ⁰C. for 30 min and loaded on a Dowex AG1-X8 column, washed and eluted with 1 M ammonium formate and 0.1 M formic acid. The eluate is counted in a scintillation counter. Data from PI hydrolysis assay are plotted using non-linear least square regression by the Prism program (Graphpad, GraphPad Software, San Diego, Calif.), from which dose ratio is also calculated. The Schild linear plot is generated from the dose-ratios obtained in four independent experiments by linear regression, and the X-intercept is used to determine the affinity of the antagonist.

Castrate Animal Studies

Studies of castrate animals provide a sensitive in vivo assay for the effects of GnRH antagonist (Andrology 25: 141-147, 1993). GnRH receptors in the pituitary gland mediate GnRH-stimulated LH release into the circulation. Castration results in elevated levels of circulating LH due to reduction of the negative feedback of gonadal steroids resulting in enhancement of GNRH stimulated LH release. Consequently, measurement of suppression of circulating LH levels in castrated macaques can be used as a sensitive in vivo measure of GNRH antagonism. Therefore, male macaques are surgically castrated and allowed to recover for four-weeks at which point elevated levels of LH are present. Animals are then administered the test compound as an oral or i.v. dose and serial blood samples taken for measurement of LH. LH concentrations in serum from these animals can be determined by immunoassay or bioassay techniques (Endocrinology 107: 902-907, 1980).

Preparation of GnRH Radioligand

The GnRH analog is labeled by the chloramine-T method. To 10 g of peptide in 20 μl of 0.5 M sodium phosphate buffer, pH 7.6, is added 1 mCi of Na¹²⁵I, followed by 22.5 μg chloramine-T in 15 μl 0.05 M sodium phosphate buffer and the mixture is vortexed for 20 sec. The reaction is stopped by the addition of 60 μg sodium metabisulfite in 30 μl 0.05M sodium phosphate buffer and the free iodine is removed by passing the reaction mixture through a C-8 Sep-Pak cartridge (Millipore Corp., Milford, Mass.). The peptide is eluted with a small volume of 80% acetonitrile/water. The recovered labeled peptide is further purified by reverse phase HPLC on a Vydac C-18 analytical column (The Separations Group, Hesperia, Calif.) on a Beckman 334 gradient HPLC system using a gradient of acetonitrile in 0.1% TFA. The purified radioactive peptide is stored in 0.1% BSA/20% acetonitrile/0.1% TFA at —80° C. and can be used for up to 4 weeks.

RIA of LH and FSH

For determination of the LH levels, each sample medium is assayed in duplicates and all dilutions are done with RIA buffer (0.01 M sodium phosphate buffer/0.15 M NaCl/1% BSA/0.01% NaN3, pH 7.5) and the assay kit is obtained from the Nation Hormone and Pituitary Program supported by NIDDK. To a 12×75 mm polyethylene test tube is added 100 μl of sample medium diluted 1:5 or rLH standard in RIA buffer and 100 μl of [125I ]-labeled rLH (˜30,000 cpm) plus 100 μl of rabbit anti-rLH antibody diluted 1:187,500 and 100 μl RIA buffer. The mixture is incubated at room temperature over-night. In the next day, 100 μl of goat anti-rabbit IgG diluted 1:20 and 100 μl of normal rabbit serum diluted 1:1000 are added and the mixture incubated for another 3 hr at room temperature. The incubated tubes are then centrifuged at 3,000 rpm for 30 min and the supernatant removed by suction. The remaining pellet in the tubes is counted in a gamma-counter. RIA of FSH is done in a similar fashion as the assay for LH with substitution of the LH antibody by the FSH antibody diluted 1:30,000 and the labeled rLH by the labeled rFSH.

Activity of GnRH Receptor Antagonists

Activity of GnRH receptor antagonists are typically calculated from the IC₅₀ as the concentration of a compound necessary to displace 50% of the radiolabeled ligand from the GnRH receptor, and is reported as a “Kᵢ” value calculated by the following equation:

$${K}_{i} = \frac{{IC}_{50}}{1 + L / {K}_{D}}$$

where L = radioligand and KD = affinity of radioligand for receptor (Cheng and Prusoff, Biochem. Pharmacol. 22:3099, 1973). GnRH receptor antagonists of this invention have a Kᵢ of 100 μM or less at the GnRH receptor. In a preferred embodiment of this invention, the GnRH receptor antagonists have a Kᵢ of less than 10 μM, and more preferably less than 1 μM, and even more preferably less than 0.1 μM (i.e., 100 nM). All of the specifically disclosed compounds set forth in Examples 1-11 were found to have a Kᵢ at the GnRH receptor of less than 1 μM.

The ability of the GnRH antagonists to inhibit the major drug metabolizing enzymes in the human liver, namely, CYP2D6 and CYP3A4, can be evaluated in vitro according to a microtiter plate-based fluorimetric method described by Crespi et al. (Anal. Biochem. 248:188-190; 1997). AMMC (i.e., 3-[2-(N,N-Diethyl-N-methylammonium)ethyl]-7-methoxy-4-methylcoumarin) and BFC (i.e., 7-benzyloxy-4-(trifluoromethyl)coumarin) at a concentration equal to Km (that is, the concentration of substrate that produces one half of the maximal velocity) are used as marker substrates for CYP2D6 and CYP3A4, respectively. Briefly, recombinant CYP2D6 or CYP3A4 is incubated with marker substrate and NADPH generating system (consisting of 1 mM NADP+, 46 mM glucose-6-phosphate and 3 units/mL glucose-6-phosphate dehydrogenase) at 37° C., in the absence or presence of 0.03, 0.09, 0.27, 0.82, 2.5, 7.4, 22, 67 and 200 μM of a sample GnRH antagonist. Reactions are stopped by the addition of an equal volume of acetonitrile. The precipitated protein is removed by centrifugation and the clear supernatant fluid is analyzed using a microtiter plate fluorimeter. GnRH antagonists of the present invention preferably have Kᵢ's greater than 250 nM at the CYP enzymes, more preferably greater than 1 μM and most preferably greater than 5 μM.

As mentioned above, the GnRH receptor antagonists of this invention have utility over a wide range of therapeutic applications, and may be used to treat a variety of sex-hormone related conditions in both men and women, as well as mammals in general. For example, such conditions include endometriosis, uterine fibroids, polycystic ovarian disease, hirsutism, precocious puberty, gonadal steroid-dependent neoplasia such as cancers of the prostate, breast and ovary, gonadotrophe pituitary adenomas, sleep apnea, irritable bowel syndrome, premenstrual syndrome, benign prostatic hypertrophy, contraception and infertility (e.g., assisted reproductive therapy such as iii vitro fertilization).

The compounds of this invention may also be useful as an adjunct to treatment of growth hormone deficiency and short stature, and for the treatment of systemic lupus erythematosis.

In addition, the compounds may be useful in combination with androgens, estrogens, progesterones, and antiestrogens and antiprogestogens for the treatment of endometriosis, fibroids, and in contraception, as well as in combination with an angiotensin-converting enzyme inhibitor, an angiotensin II-receptor antagonist, or a renin inhibitor for the treatment of uterine fibroids. The compounds may also be used in combination with bisphosphonates and other agents for the treatment and/or prevention of disturbances of calcium, phosphate and bone metabolism, and in combination with estrogens, progesterones and/or androgens for the prevention or treatment of bone loss or hypogonadal symptoms such as hot flashes during therapy with a GnRH antagonist.

For the purposes of administration, the compounds of the present invention may be formulated as pharmaceutical compositions. Pharmaceutical compositions comprise one or more compounds of structure (I) in combination with a pharmaceutically acceptable carrier and/or diluent. Such compound is present in the composition in an amount which is effective to treat the particular disorder or interest—for example, in an amount sufficient to achieve the desired GnRH receptor antagonist activity, and preferably with acceptable toxicity to the patient. Typically, the pharmaceutical compositions of the present invention may include the compound(s) of structure (I) in an amount from 0.1 mg to 250 mg per dosage depending upon the route of administration, and more typically from 1 mg to 60 mg. Appropriate concentrations and dosages can be readily determined by one skilled in the art.

Pharmaceutically acceptable carrier and/or diluents are familiar to those skilled in the art. For compositions formulated as liquid solutions, acceptable carriers and/or diluents include saline and sterile water, and may optionally include antioxidants, buffers, bacteriostats and other common additives. The compositions can also be formulated as pills, capsules, granules, or tablets which contain, in addition to a GnRH receptor antagonist, diluents, dispersing and surface active agents, binders, and lubricants. One skilled in this art may further formulate the GnRH receptor antagonist in an appropriate manner, and in accordance with accepted practices, such as those disclosed in Remington's Pharmaceutical Sciences, Gennaro, Ed., Mack Publishing Co., Easton, Pa. 1990.

In another embodiment, the present invention provides a method for treating sex-hormone related conditions as discussed above. Such methods include administration of a compound of the present invention to a warm-blooded animal in an amount sufficient to treat the condition. In this context, “treat” includes prophylactic administration. Such methods include systemic administration of a GnRH receptor antagonist of this invention, preferably in the form of a pharmaceutical composition as discussed above. As used herein, systemic administration includes oral and parenteral methods of administration. For oral administration, suitable pharmaceutical compositions of GnRH receptor antagonists include powders, granules, pills, tablets, and capsules as well as liquids, syrups, suspensions, and emulsions. These compositions may also include flavorants, preservatives, suspending, thickening and emulsifying agents, and other pharmaceutically acceptable additives. For parental administration, the compounds of the present invention can be prepared in aqueous injection solutions which may contain, in addition to the GnRH receptor antagonist, buffers, antioxidants, bacteriostats, and other additives commonly employed in such solutions.

The following examples are provided for purposes of illustration, not limitation. In summary, the GNRH receptor antagonists of this invention may be assayed by the general methods disclosed above, while the following Examples disclose the synthesis of representative compounds of this invention.

### EXAMPLES

HPLC Methods for analyzing the samples

Retention time, t_{R}, in minutes

### Method 1 (HPLC-MS)

### Column: Waters ODS-AQ, 2.0×50 mm

Mobile phase: A=water with 0.05% trifluoroacetic acid; B=acetonitrile with 0.05% trifluoroacetic acid
Gradient: 95% A/ 5%B to 5%A/95%B over 13.25 min and hold 5%A/95%B over 2 min then return to 95%A/5%B over 0.25 min.
Flow Rate: 1 mL/min
UV wavelength: 220 and 254 nM

### Method 2 (HPLC-MS)

### Column: Waters ODS-AQ, 2.0×50 mm

Mobile phase: A=water with 0.05% trifluoroacetic acid; B=acetonitrile with 0.05% trifluoroacetic acid
Gradient: 95% A/5%B to 10%A/90%B over 2.25 min and hold 10%A/90%B over 1.0 min then return to 95%A/5%B over 0.1 min.
Flow Rate: 1 mL/min
UV wavelength: 220 and 254 nM

### Method 3 (Analytical HPLC-MS)

Column: XTerra MS, C₁₈, 5 μ, 3.0×250 mm cartridge
Mobile phase: A=water with 0.025% trifluoroacetic acid; B=acetonitrile with 0.025% trifluoroacetic acid
Gradient: 5% B/95% A to 90% B/10% A over 47.50 minutes, maintaining 99% for 8.04 minutes.
Flow Rate: 1 mL/min
UV wavelength: 220 and 254 nM

### EXAMPLE 1

### 5-BROMO-1-[2-FLUORO-6-(TRIFLUOROMETHYL)BENZYL]-6-METHYLPYRIMIDINE-2,4(1 H,3H)-DIONE

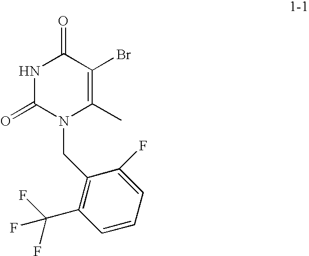

Step 1A: Preparation of 2-fluoro-6-(trifluoromethyl)benzylamine 1a

To 2-fluoro-6-(trifluoromethyl)benzonitrile (45 g, 0.238 mmol) in 60 mL of THF was added 1 M BH₃:THF slowly at 60° C. and the resulting solution was refluxed overnight. The reaction mixture was cooled to ambient temperature. Methanol (420 mL) was added slowly and stirred well. The solvents were then evaporated and the residue was partitioned between EtOAc and water. The organic layer was dried over Na₂SO₄. Evaporation gave la as a yellow oil (46 g, 0.238 mmol). MS (Cl) m/z 194.0 (MH⁺).

Step 1B: Preparation of N-[2-fluoro-6-(trifluoromethyl)benzyl]urea 1b

To 2-fluoro-6-(trifluoromethyl)benzylamine la (51.5 g, 0.267 mmol) in a flask, urea (64 g, 1.07 mmol), HCl (conc., 30.9 mmol, 0.374 mmol) and water (111 mL) were added. The mixture was refluxed for 6 hours. The mixture was cooled to ambient temperature, further cooled with ice and filtered to give a yellow solid. Recrystallization with 400 mL of EtOAc gave 1b as a white solid (46.2 g, 0.196 mmol). MS (CI) m/z 237.0 (MH⁺).

Step 1C: Preparation of 1-[2-fluoro-6-(trifluoromethyl)benzyl]-6-methylprimidine-2,4(1H,3H)-dione 1c

NaI (43.9 g, 293 mmol) was added to N-[2-fluoro-6-(trifluoromethyl)benzyl]urea 1b (46.2 g, 19.6 mmol) in 365 mL of acetonitrile. The resulting mixture was cooled in an ice-water bath. Diketene (22.5 mL, 293 mmol) was added slowly via dropping funnel followed by addition of TMS (37.2 mL, 293 mmol) in the same manner. The resulting yellow suspension was allowed to warm to room temperature slowly and was stirred for 20 hours. LC-MS showed the disappearance of starting material. To the yellow mixture 525 mL of water was added and stirred overnight. After another 20 hours stirring, the precipitate was filtered via Buchnner funnel and the yellow solid was washed with water and EtOAc to give 1c as a white solid (48.5 g, 16 mmol). ¹ H NMR (CDCl₃) δ2.15 (3H, s), 5.37 (2H, s), 5.60 (1H, s), 7.23-7.56 (3H, m), 9.02 (1H, s); MS (CI) m/z 303.0 (MH⁺).

Step 1D: Preparation of 5-bromo-1-[2-fluoro-6-(trifluoromethyl)benzyl]-6-methylprimidine-2,4(1H,3H)-dione 1-1

Bromine (16.5 mL, 0.32 mmol) was added to 1-[2-fluoro-6-(trifluoromethyl)benzyl]-6-methylpyrimidine-2,4(1H,3H)-dione 1c (48.5 g, 0.16 mol) in 145 mL of acetic acid. The resulting mixture became clear then formed precipitate within an hour. After 2 hours stirring, the yellow solid was filtered and washed with cold EtOAc to an almost white solid. The filtrate was washed with sat. NaHCO₃ and dried over Na₂SO₄. Evaporation gave a yellow solid which was washed with EtOAC to give a light yellow solid. The two solids were combined to give 59.4 g of 1-1 (0.156 mol) total. ¹H NMR (CDCl₃) δ2.4 (3H, s), 5.48 (2 H, s), 7.25-7.58 (3 H, m), 8.61 (1 H, s); MS (CI) m/z 380.9 (MH⁺).

### EXAMPLE 2

### 5-BROMO-1-[2-FLUORO-6-(TRIFLUOROMETHYL)BENZYL]PYRIMIDINE-2,4(1 H,3H)-DIONE

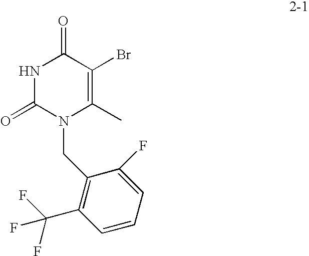

Step 2A: Preparation of 5-bromo-1-[2-fluoro-6-(trifluoromethyl)benzyl]pyrimidine-2,4(1 H,3 H)-dione 2-1

A suspension of 5-bromouracil (3.1 g, 16.2 mmol) in 100 mL of anhydrous acetonitrile is treated with N,O-bis(trimethylsilyl)acetamide (8 mL, 32.4 mmol). The reaction mixture is heated at 80° C. under nitrogen for 2 hours. The solution is cooled to ambient temperature and a solution of 2-fluoro-6-(trifluoromethyl)benzyl bromide (5.0 g, 19.4 mmol) in acetonitrile (20 mL) is added and the reaction mixture is heated overnight under nitrogen. The reaction is cooled, quenched with MeOH, and partitioned between dichloromethane and water. The organic layer is washed with brine, dried (sodium sulfate), and evaporated to give a solid. The crude product is triturated with ether, filtered, and washed with ether three times providing 5-bromo-1-[2-fluoro-6-(trifluoromethyl)benzyl]pyrimidine-2,4(1 H,3 H)-dione 2-1. NMR (CDCl₃) δ8.72 (s, 1 H), 7.64-7.37 (3 H, m), 7.21 (1 H, s), 5.18 (s, 2 H), MS (CI) m/z 366.8, 368.8 (MH⁺).

### EXAMPLE 3

### 5-(2-{1-AMINO-2-[5-(2-FLUORO-3-METHOXY-PHENYL)-3-(2-FLUORO-6-TRIFLUOROMETHYL-BENZYL)-4-METHYL-2,6- DIOXO-3,6-DIHYDRO-2H-PYRIMIDIN- 1-YL]-ETHYL}-PHENOXY)-PENTANOIC ACID

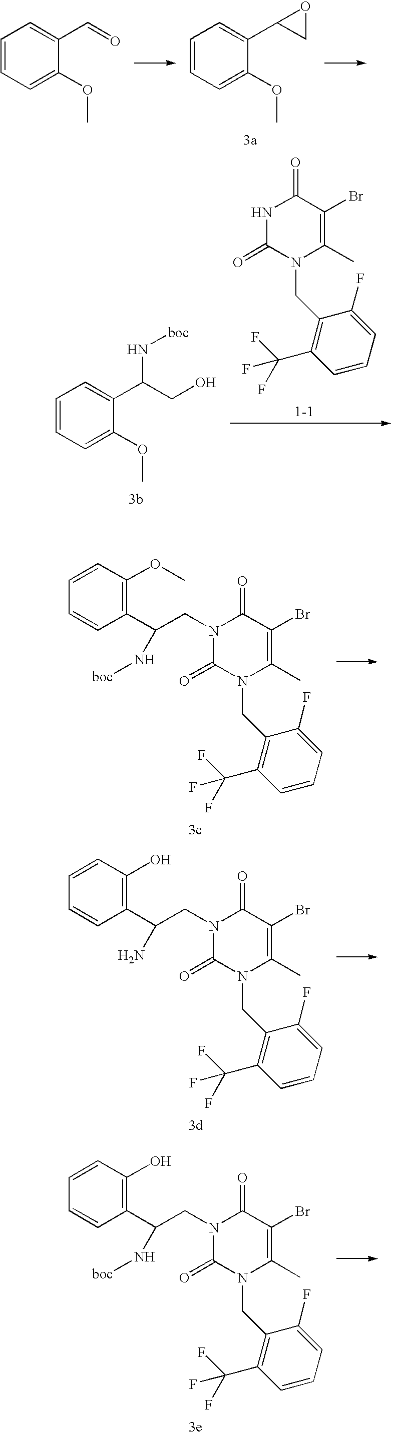

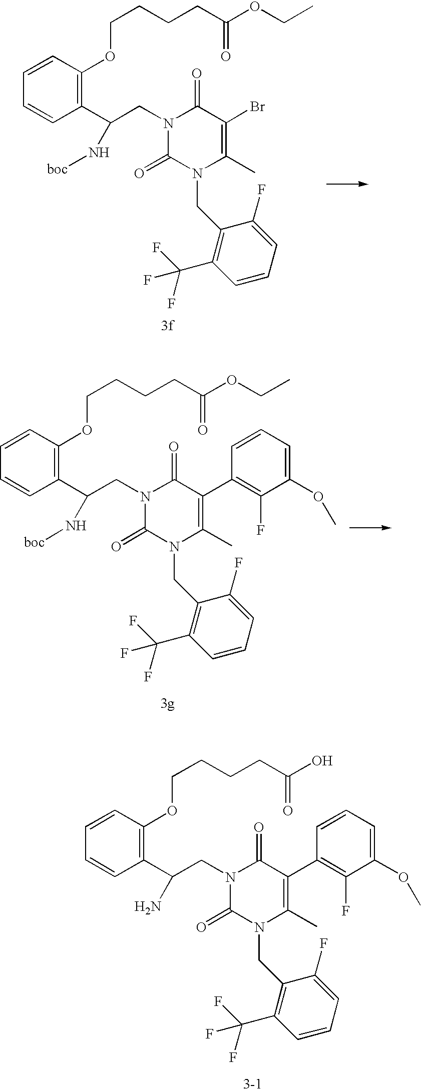

### Step 3A:

A mixture of o-anisaldehyde (10 g, 73.4 mmol), trimethylsulfonium iodide (18 g, 88.1 mmol), and tetrabutyl ammonium iodide (271 mg, 0.734 mmol) in dichloromethane (250 mL)/ aqueous NaOH (50%, 165 mL) was stirred at room temperature for 1 week. After dilution with water, the organic layer was separated and washed with water and brine. The organic layer was dried over MgSO₄, filtered and concentrated to yield the epoxide 3a (10.3 g).

### Step 3B:

To the epoxide 3a (1.39 g, 9.27 mmol) in acetone/H₂O (20/20 mL) was added sodium azide (904 mg, 13.9 mmol) and the mixture was refluxed for 3 hours. Acetone was removed by evaporation and the aqueous solution was extracted with dichloromethane. The organic layer was dried over MgSO₄ and concentrated to yield the crude azide, which was redissolved in EtOH (20 mL). Palladium on carbon (10%, 100 mg) was added and the mixture was stirred overnight at room temperature under hydrogen atmosphere. The resulting mixture was filtered through Celite and was concentrated to yield an oil which was dissolved in dichloromethane (20 mL). Di-tert-butyldicarbonate (2.43 g, 11.1 mmol) was added. The mixture was stirred at room temperature overnight, and concentrated to yield 3b as a yellow oil (1.1 g). NMR (CDCl₃), δ7.30-7.22 (2 H, m), 6.98-6.88 (2 H, m), 5.58 (1 H, brs), 5.07 (1 H, brs), 3.85 (3 H, s), 3.82 (2 H, brs), 1.44 (9 H, s).

### Step 3C:

To the Boc-amino alcohol 3b (847 mg, 3.15 mmol) and 5-bromo-1-[2-fluoro-6-(trifluoromethyl)benzyl]-6-methylpyrimidine-2,4(1 H,3 H)-dione 1-1 (800 mg, 2.1 mmol) in dry THF (15 mL) were added diisopropyl azodicarboxylate (DIAD, 0.62 mL, 15 mmol) and triphenylphosphine (826 mg, 3.15 mmol). The mixture was stirred overnight, concentrated and purified by silica gel chromatography (EtOAc/Hexane) to yield 3c (1.78 g, which contains some DIAD related side product). MS (CI) m/z 530.2/532.2 (MH⁺), HPLC: t_{R}=2.7 min (Method 2).

Step 3D:

To 3c in dichloromethane (15 mL) at —70 ° C., boron tribromide (BBr₃, 1 M in dichloromethane, 14 mL, 14 mmol) was added slowly. The mixture was then stirred overnight while the reaction slowly warmed to room temperature. The mixture was concentrated to remove the volatiles and was dissolved in dichloromethane (30 mL), washed with sat. NaHCO₃ solution and brine, dried over MgSO₄ and concentrated to give 3d (1.2 g). NMR (CDCl₃), δ, 7.57 (1 H, d, J=7.5 Hz), 7.48-7.41 (1 H, m), 7.29-7.23 (1 H, m), 7.17-7.09 (2 H, m), 7.02-6.94 (1 H, m), 6.77 (1 H, t, J=7.5 Hz), 5.59 (1 H, d, J=13.2 Hz), 5.40 (1 H, d, J=13.2 Hz), 4.66-4.56 (2 H, m), 4.24-4.14 (1 H, m), 2.43 (3 H, s). MS (CI) m/z 515.9/517.9 (MH⁺), HPLC: t_{R}=2.2 min (Method 2).

Step 3E:

To 3d (1.2 g) in dichloromethane (10 mL), was added triethyl amine (0.67 mL, 4.8 mmol), followed by di-tert-butyldicarbonate (692 mg, 3.2 mmol). The mixture was stirred at room temperature for 1 day. Dichloromethane (100 mL) and water (50 mL) were added. The organic layer was separated, washed with brine, dried over MgSO₄, and concentrated to yield a solid. The solid was washed with dichloromethane (2×5 mL) to yield 3e as a white solid (1.0 g). NMR (CDCl₃) δ7.90 (1 H, brs), 7.50 (1 H, d, J=7.8 Hz), 7.37 (1 H, dd, J=7.8, 12.6 Hz), 7.19-7.07 (3 H, m), 6.88 (1 H, d, J=7.1 Hz), 6.81 (1 H, m), 5.66-5.57 (2 H, m), 5.35 (1 H, d, J=17.4 Hz), 5.17-5.11 (1 H, m), 4.70-4.60 (1 H, m), 3.98 (1 H, dd, J=2.7, 13.2 Hz), 2.39 (3 H, s), 1.28 (9 H, s).

Step 3F:

To the phenol 3e (188.2 mg, 0.31 mmol) in dry DMF (2 mL) was added methyl bromovalerate (90.7 mg, 0.465 mmol), followed by K₂CO₃ (64.3 mg, 0.465 mmol) and the mixture was heated at 100° C. in a microwave for 3 minutes. Ethyl acetate (10 mL) was added, the organic solution was then washed with H₂O, brine, dried over MgSO₄, and purified by prep TLC plate (hexane/EtOAc=3/2) to give 144 mg of 3f. MS (CI) m/z 629.9/631.8 (MH⁺), HPLC: t_{R}=2.90 min (Method 2).

Step 3G:

To 3f (144 mg, 0.2 mmol) in a sealable tube was added 2-fluoro-3-methoxyphenyl boronic acid (67 mg, 0.4 mmol), Na₂CO₃ (125.3 mg, 1.18 mmol) and dioxane/H₂O (9/1, 3 mL). Nitrogen was bubbled through the mixture for 10 min, then tetrakis(triphenylphosphine)palladium(0) (Pd(PPh₃)₄, 22.8 mg, 0.02 mmol) was added. The tube was sealed and heated at 100° C. overnight. The mixture was extracted with ethyl acetate (20 mL), the organic layer washed with water and brine, dried over MgSO₄, and concentrated to give 3g as an oil. MS (CI) m/z 676.0 (MH⁺), HPLC: t_{R}=2.94 min (Method 2).

Step 3H:

Compound 3g was dissolved in THF (2 mL) and LiOH (120 mg, 5 mmol) was added, followed by 5 drops of water. The mixture was stirred at 60° C. overnight. Sat. NaHSO₄ was then added to acidify the mixture. The crude product was extracted with ethyl acetate (10 mL). The ethyl acetate layer was washed with water, brine and was concentrated. TFA/dichloromethane (1/1, 2 mL) was added and the mix was stirred at room temperature for 1 hr, concentrated and the mixture was purified by prep. TLC to yield 65 mg of 3-1. NMR (CDCl₃/DMSO-d₆=7/3, rotamers were observed), d, 8.37 (2 H, br), 7.28-7.25 (2 H), 7.20-7.13 (1 H, m), 7.06-6.96 (3 H, m), 6.85-6.79 (1 H, m), 6.73-6.47 (3 H, m), 5.28-5.20 (1 H, 2d, J=17.4 Hz), 5.03-4.95 (1 H, 2d, J=17.4 Hz), 4.68-4.56 (1 H, m), 4.48-4.33 (1 H, m), 4.08-3.86 (1 H, m), 3.80-3.66 (2H, m), 3.60, 3.59 (3 H, 2 s), 2.32-2.28 (2 H, m), 2.08-2.00 (2 H, m), 1.77 (3H, s), 1.66- 1.45 (4 H, 2 m).

The following compounds were made using the above procedures:

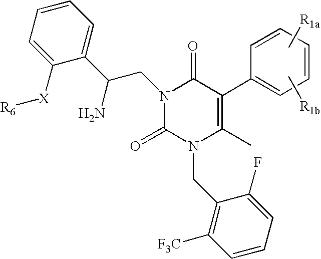

| Ex | —X—R₆ | 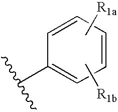 | MW | MH⁺ | t_{R}(min) | HPLCMethod |
|---|---|---|---|---|---|---|
| 3-1 | 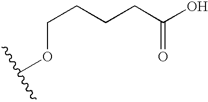 | 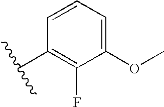 | 661.62 | 662.0 | 5.18 | 1 |
| 3-2 | 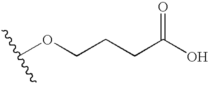 | 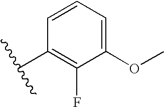 | 647.59 | 648.0 | 4.98 | 1 |
| 3-3 | 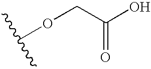 | 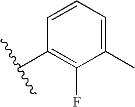 | 603.54 | 603.9 | 4.99 | 1 |
| 3-4 | 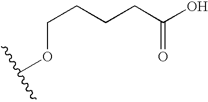 | 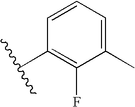 | 645.62 | 646.0 | 5.26 | 1 |
| 3-5 | 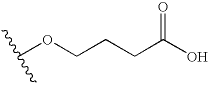 | 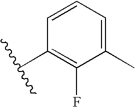 | 631.60 | 632.0 | 5.06 | 1 |
| 3-6 | 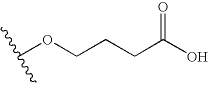 | 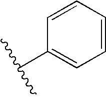 | 599.58 | 600.5 | 6.02 | 1 |
| 3-7 | 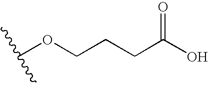 | 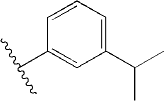 | 641.66 | 642.1 | 6.28 | 1 |
| 3-8 | 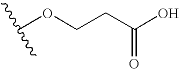 | 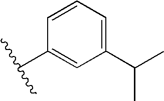 | 627.63 | 628.1 | 28.95 | 3 |
| 3-9 | 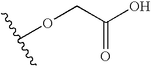 | 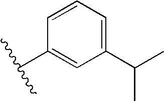 | 613.61 | 614.1 | 6.39 | 1 |
| 3-10 | 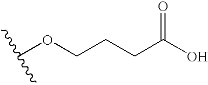 | 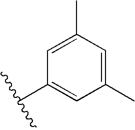 | 627.63 | 628.1 | 6.47 | 1 |
| 3-11 | 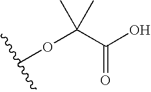 | 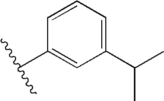 | 641.66 | 642.1 | 7.18 | 1 |
| 3-12 | 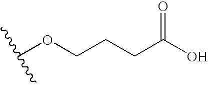 | 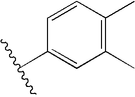 | 627.63 | 628.0 | 6.60 | 1 |
| 3-13 | 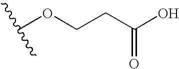 | 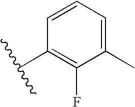 | 617.57 | 618.0 | 5.19 | 1 |
| 3-14 | 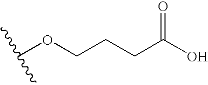 | 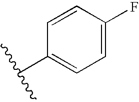 | 617.57 | 618.0 | 4.77 | 1 |

### EXAMPLE 4

### (2-{(S)-1-AMINO-3-[3-(2-FLUORO-6-TRIFLUOROMETHYL-BENZYL)-4-METHYL-2,6-DIOXO-5-PHENYL-3,6-DIHYDRO-2H-YRIMIDIN-1-YL]-PROPYL}-PHENOXY)-ACETIC ACID

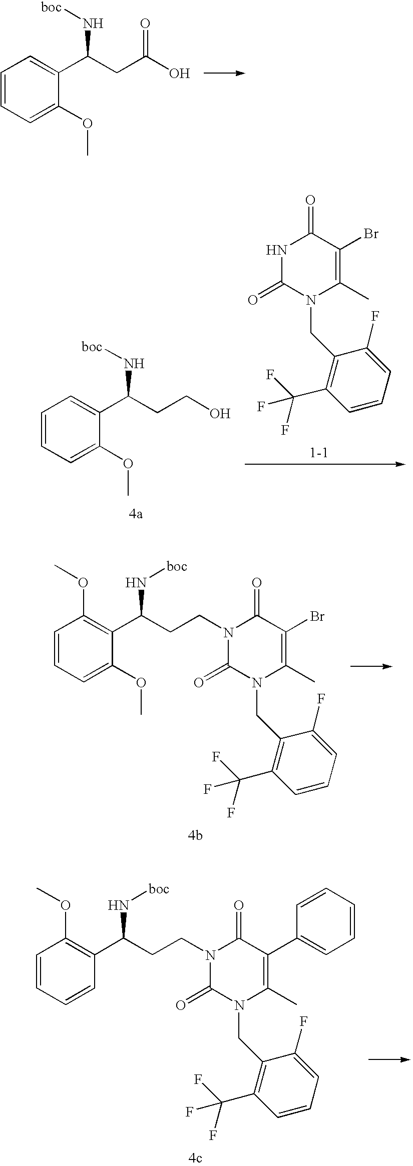

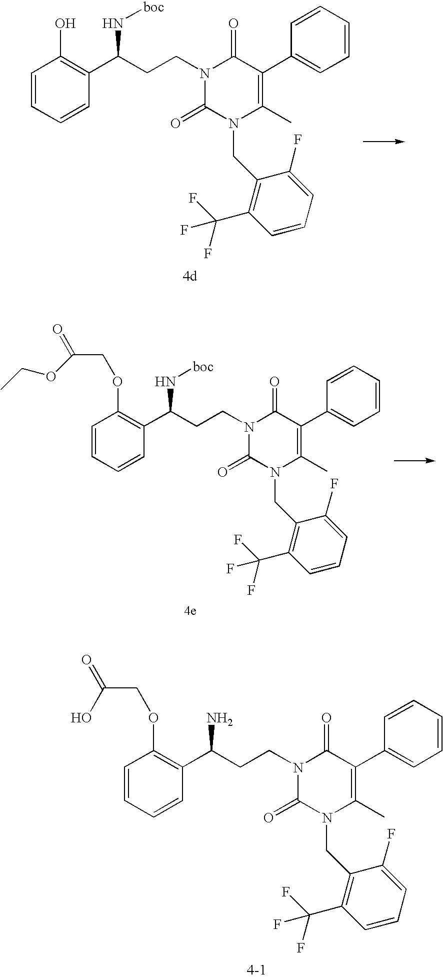

### Step 4A:

1M BH₃ in THF (34 mL) was added slowly to a solution of Boc-(S)-3-amino-3-(2-methoxyphenyl)-propionic acid (1.0 g, 3.39 mmol) in dry THF (10 mL) at −78° C. The reaction mixture was allowed to warm to room temperature and was stirred overnight. The mixture was poured into an aqueous 6N NaHSO₄ solution (20 mL). The mixture was extracted with ethyl acetate and the organic layer was separated and washed with water and brine, dried over MgSO₄ and concentrated to give 4a as an oil. MS (CI) m/z 165.2 (MH⁺), HPLC: t_{R}=2.56 min (Method 2).

### Step 4B:

To oil 4a in dry THF (15 mL) was added the 5-bromo-1-[2-fluoro-6-(trifluoromethyl)benzyl]-6-methylpyrimidine-2,4(1 H,3 H)-dione 1-1 (1.18 g, 3.1 mmol), followed by addition of triphenylphosphine (1.22 g, 4.65 mmol) and diisopropyl azodicarboxylate (0.92 mL, 4.65 mmol). The mixture was stirred at room temperature for 3 hours, then was concentrated and dissolved in ethyl acetate (100 mL). The ethyl acetate layer was washed with water and brine, dried over MgSO₄ and concentrated. Purification by silica gel chromatography (hexane/EtOAc 3/2 as elutant) gave 1.26 g of 4b. MS (CI) in/z 544.1/546.1 (MH⁺), HPLC: t_{R}=3.11 min (Method 2).

Step 4C:

To 4b (77 mg, 1.2 mmol) in a sealable tube containing dioxane/water (9/1, 5 mL) was added phenylboronic acid (294 mg, 2.4 mmol) and Na₂CO₃ (763 mg, 7.2 mmol). Nitrogen was bubbled through the mixture for 5 minutes and Pd(PPh₃)₄ (138.6 mg, 0.12 mmol) was then added. The tube was sealed and heated at 100° C. with stirring overnight. The mixture was extracted with ethyl acetate (100 mL), the organic layer washed with water and brine, dried over MgSO₄ and concentrated. The residue was purified by silica gel column chromatography to yield 677 mg of 4c. MS (CI) m/z 542.3 (MH⁺), HPLC: t_{R}=3.23 min (Method 2).

Step 4D:

BBr₃ (5.28 mL, 5.28 mmol, 1 M in dichloromethane) was added slowly to 4c (677 mg, 1.06 mmol) in dichloromethane (5 mL) at κ78° C. The mixture was then stirred at room temperature overnight. The solvent and excess of BBr₃ were removed by evaporation, the residue was dissolved in MeOH and evaporation yielded an oil which was dissolved in dichloromethane. Trimethylamine was added to neutralize the excess HBr (until pH=7) and di-tert-butyldicarbonate (255 mg, 1.17 mmol) was added. The mixture was stirred at room temperature for 2 days. The mixture was concentrated and water /EtOAc (10 mL/100 mL) was added. The organic layer was separated, washed with water and brine, dried over MgSO₄, and concentrated to give 4d (450 mg). MS (CI) m/z 528.4 (MH⁺), HPLC: t_{R}=3.05 min (Method 2).

Step 4E:

To 4d (100 mg, 0.159 mmol) in DMF (1.5 mL) was added ethyl bromoacetate (0.027 mL, 0.24 mmol) and K₂CO₃ (33 mg, 0.24 mmol). The mixture was heated in a microwave at 100 ° C. for 3 minutes. The mixture was diluted with ethyl acetate (20 mL) and water (10 mL). The organic layer was separated, washed with water and brine and dried over MgSO₄. Concentration gave compound 4e as an oil (85 mg). MS (CI) m/z 614.0 (MH⁺)

Step 4F:

To the ester 4e (85 mg) in THF (2 mL), was added LiOH (120 mg, 0.5 mmol), followed by 5 drops of water. The mixture was heated at 50° C. overnight, acidified by NaHSO₄, and extracted with ethyl acetate (10 mL). The ethyl acetate layer was evaporated and the residue was treated with dichloromethane/TFA (1/1, 3 mL) at room temperature for 1 hour. Concentration and purification by prep TLC plate gave 27 mg of 4-1. NMR (sodium salt, CDCl₃), δ, 7.46 (1 H, d, J=7.8 Hz), 7.36-7.16 (6 H, m), 7.11 (2 H, d, J=6.9 Hz), 6.98-6.94(1 H, m), 6.75 (1 H, t, J=7.5 Hz), 6.65 (1 H, d, J=8.4 Hz), 5.39 (2 H, s), 4.17 (2 H, s), 4.00-3.73 (3 H, m), 2.25-1.87 (2 H, 2m), 1.94 (3 H, s).

The following compounds were made using the above procedures:

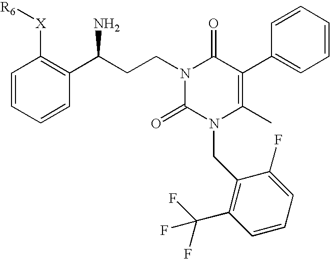

| | | | | t_{R} (min) |
|---|---|---|---|---|
| Ex | —X—R₆ | MW | MH⁺ | Method 1 |
| 4-1 | 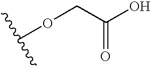 | 585.55 | 586 | 6.04 |
| 4-2 | 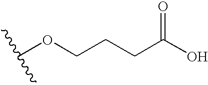 | 613.61 | 614 | 6.00 |
| 4-3 | 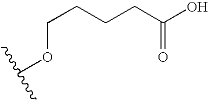 | 627.63 | 628 | 6.14 |

### EXAMPLE 5

### 4-(2-{(S)-1-AMINO-3-[3-(2-FLUORO-6-TRIFLUOROMETHYL-BENZYL)-5-(3-ISOPROPYL-PHENYL)-4-METHYL-2,6-DIOXO-3,6-DIHYDRO-2H-PYRIMIDIN-1-YL]-PROPYL}-PHENOXY)-BUTYRIC ACID

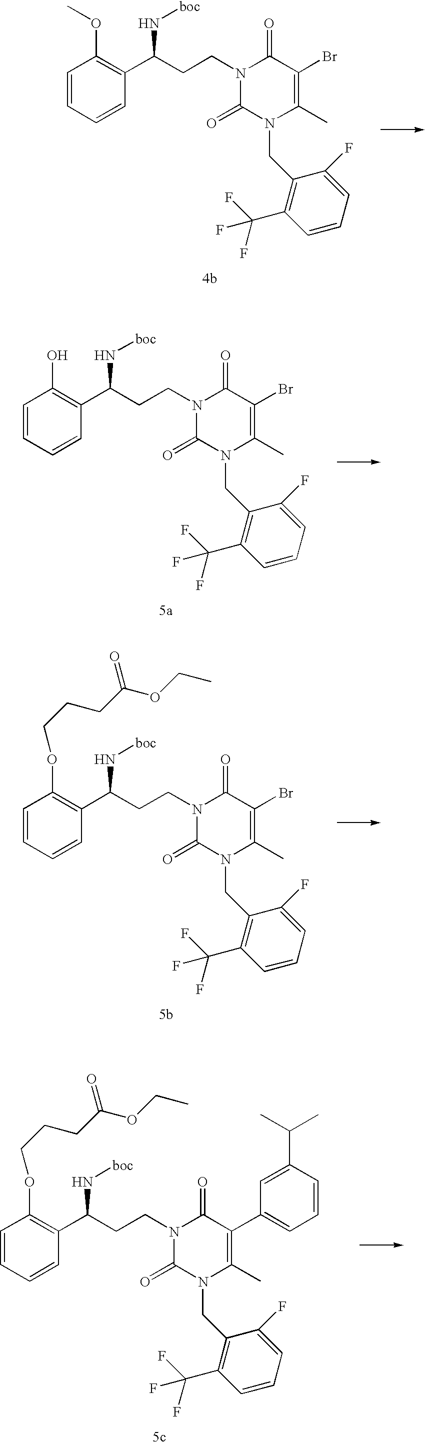

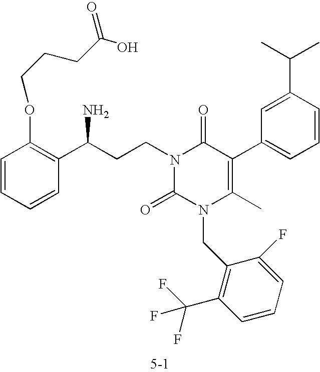

Step 5A:

To 4b (1.28 g, 2 mmol) in dichloromethane (10 mL) at −78 ° C. was added BBr₃ (1 M in dichloromethane, 9.9 mL, 9.94 mmol) slowly. The mixture was stirred at room temperature for 3 hours and the solvent was removed by evaporation. Methanol (10 mL) was added and evaporated and the residue was suspended in dichloromethane (15 mL). Triethyl amine was added until pH was 8-9, then di-tert-butyldicarbonate (0.6 mL, 2.6 mmol) was added. The mixture was stirred at room temperature overnight, washed with water (10 mL) and brine (10 mL), then dried over MgSO₄. After concentration, the residue was purified by silica gel column chromatography to give 924 mg of compound 5a. MS (CI) m/z 527.8/529.8 (MH⁺), HPLC: t_{R}=2.81 min (Method 2).

Step 5B:

To 5a (924 mg, 1.47 mmol ) in dry DMF (10 mL), was added ethyl 4-bromobutyrate (0.32 mL, 2.2 mmol) and K₂CO₃ (406 mg, 2.94 mmol) and the mixture was heated at 60° C. overnight. After cooling to room temperature, the mixture was diluted with water (20 mL) and extracted with ethyl acetate (100 mL). The ethyl acetate layer was washed with water (10 mL) and brine (10 mL), dried over MgSO₄, concentrated and purified by silica gel column chromatography (hexane/ethyl acetate 6.5/3.5) to yield 0.92 g of 5b. NMR (CDCl₃), δ, 7.55 (1 H, d, J=7.5 Hz), 7.45-7.38 (1 H, m), 7.23-7.20 (3 H, m), 6.89 (1 H, t, J=7.5 Hz), 6.83 (1 H, d, J=8.4 Hz), 5.49-5.46 (1 H, m), 5.43 (2 H, s), 5.02-4.94 (1 H, m), 4.13 (2 H, q, J=6.9 Hz), 4.10-4.00 (4 H, m), 2.55 (2 H, t, J=7.5 Hz), 2.37 (3 H, s), 2.20-2.11 (4 H, m), 1.42 (9 H, s), 1.25 (3 H, t, J=6.9 Hz). MS (CI) m/z 644.0/646.0 (MH⁺), HPLC: t_{R}=2.77 min (Method 2).

Step 5C:

To 5b (104.6 mg, 0.14 mmol) in a sealable tube containing a mixture of dioxane (1.8 mL) and water (0.2 mL), was added 3-isopropyphenyl boronic acid (45.9 mg, 0.28 mmol), followed by addition of Na₂CO₃ (89 mg, 0.84 mmol). The mixture was purged with N₂ for 5 min, then Pd(PPh₃)₄ (16.2 mg, 0.014 mmol) was added. The slurry was sealed and heated at 100° C. overnight with stirring. The mixture was then treated with ethyl acetate (20 mL) and water (10 mL). The organic layer was separated and further washed with water and brine and was dried over MgSO₄. Upon concentration, the residue was purified by prep TLC plate (hexane/ethyl acetate =3/2) to give 5c (100 mg). MS (CI) m/z 684.1 (MH⁺), HPLC: t_{R}=3.07 min (Method 2).

Step 5D:

The ester 5c (100 mg) was then treated with LiOH (120 mg, 5 mmol) in THF (2 mL) with 5 drops of water at 60° C. overnight, then was acidified by IN HCl. The mixture was extracted with ethyl acetate. The ethyl acetate solution was then washed with water, dried and concentrated to give an oil, which was stirred with a mixture of dichloromethane (1 mL) and TFA (1 mL) for 0.5 hour. The volatiles were evaporated and purification by prep TLC plate (dichloromethane /MeOH=9/1) gave 50 mg of 5-1.

The following compounds were made using the above procedures:

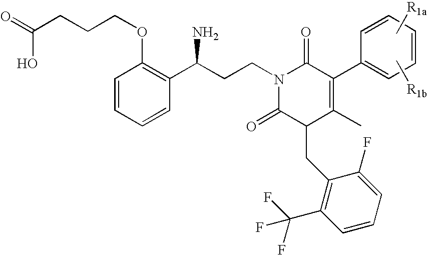

| Ex | 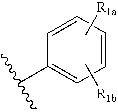 | MW | MH⁺ | t_{R} (min)Method 1 |
|---|---|---|---|---|
| 5-1 | 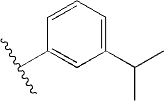 | 655.68 | 656.1 | 5.44 |
| 5-2 | 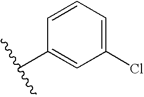 | 648.05 | 648.2 | 5.28 |
| 5-3 | 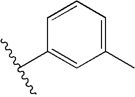 | 627.63 | 628.0 | 6.28 |
| 5-4 | 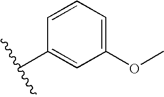 | 643.63 | 644.0 | 6.03 |
| 5-5 | 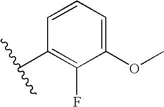 | 661.62 | 662.3 | 5.14 |

### EXAMPLE 6

### 3-(2-{(S)-1-AMINO-3-[3-(2-FLUORO-6-TRIFLUOROMETHYL-BENZYL)-4-METHYL-2,6-DIOXO-5-PHENYL-3,6-DIHYDRO-2H-PYRIMIDIN- 1-YL]-PROPYL} -PHENOXY)-PROPIONIC ACID

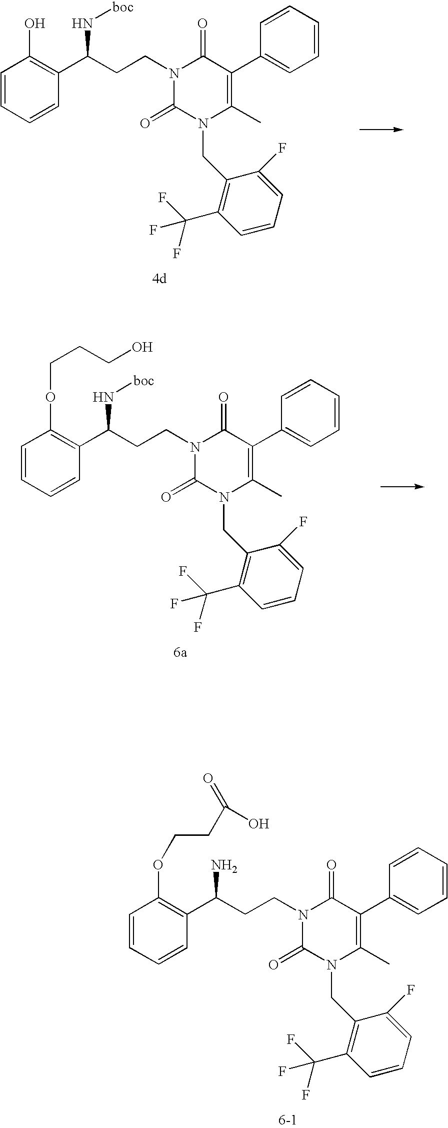

Step 6A:

To 4d (290 mg, 0.46 mmol) in DMF (3 mL), was added 3-bromo-1-propanol (0.627 mL, 0.694 mmol), followed by addition of K₂CO₃ (192 mg, 1.389 mmol). After the mixture was stirred at room temperature overnight, water (5 mL) and ethyl acetate (20 mL) were added. The organic layer was separated and washed with water, and brine and was dried over MgSO₄. After concentration, the residue was purified by prep TLC plate (hexane/ethyl acetate =3/2) to give 202 mg of compound 6a. MS (CI) m/z 681.1 (MH⁺), HPLC: t_{R}=2.90 min (Method 2).

Step 6B:

To 6a (168 mg, 0.245 mmol) in a solution of acetonitrile/NaH₂PO₄ buffer (3/2, pH=6.1, 4 mL) at 45° C., was added TEMPO (2,2,6,6-tetramethylpiperdinyloxy, free radical, 3.8 mg, 0.024 mmol), a solution of sodium chlorite (44.36 mg, 2.0 eq.) in water (0.245 mL) and a solution of sodium hypochlorite (0.013 mL, 6.0 eq.) in water (0.245 mL). The mixture was stirred at 45° C. overnight then was cooled to room temperature. Sat. Na₂SO₃ was added dropwise until the reaction mixture became colorless. Acetonitrile was evaporated and ethyl acetate (25 mL) was added. The organic layer was separated, washed with 1 N HCl, water, and brine and was dried over MgSO₄. The solvents were evaporated and the residue was stirred with dichloromethane/TFA (1/1, 2 mL) for 1 hour. Following evaporation of the volatiles, the resulting oil was purified by prep TLC plate (dichloromethane/MeOH=9/1) to give 105 mg of 6-1, which after treatment with 1 eq of NaOH in water and lyophilization to dryness gave 6-1 sodium salt. NMR (CDCl₃, 95%; DMSO-d₆, 5%), δ, 7.45 (1 H, d, J=7.8 Hz), 7.37-7.15 (7 H, m), 7.10-7.08 (2 H, m), 6.86 (1 H, d, J=7.5 Hz), 6.80 (1 H, d, J=8.4 Hz), 5.35 (2 H, s), 4.22 (1 H, t, J=7.5 Hz), 4.17-4.04 (2 H, m), 3.97-3.90 (2 H, m), 2.64-2.58 (2 H, m), 2.40-2.24 (2 H, m), 1.95 (3 H, s). MS (CI) m/z 600.0 (MH⁺), HPLC: t_{R}=3.00 min (Method 2).

### EXAMPLE 7

### 4-(2-{1-DIMETHYLAMINO-2-[5-(2-FLUORO-3-METHOXYPHENYL)-3-(2-FLUORO-6-TRIFLUOROMETHYLBENZYL)-4-METHYL-2,6-DIOXO-3,6-DIHYDRO-2H-PYRIMIDIN-1-YL]-ETHYL} -PHENOXY)-BUTYRIC ACID (7-1)

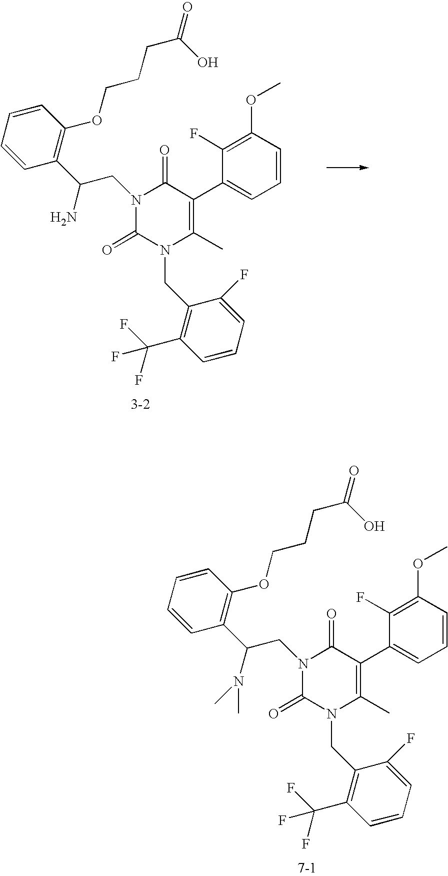

Step 7A:

To the compound 3-2 (47 mg, 0.07 mmol) in MeOH (1 mL) was added aqueous formaldehyde (54 μl, 0.7 mmol), then BH₃.pyridine (14.5 μl, 0.145 mmol). The mixture was stirred at room temperature for 1 hr, concentrated and purified by Prep-TLC plate (1% TEA/10% MeOH/dichloromethane) to give 30.2 mg of the desired product 7-1. MS (CI) m/z 676.1 (MH⁺), HPLC: t_{R}=6.06 min (Method 1).

The following compounds were made using the above procedure:

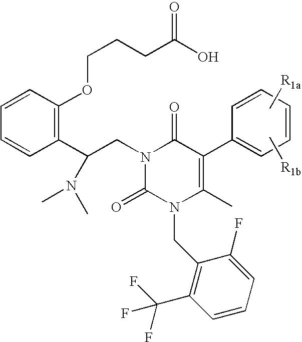

| Ex | 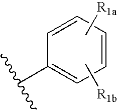 | MW | MH⁺ | t_{R} (min) | HPLCMethod |
|---|---|---|---|---|---|
| 7-1 | 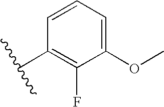 | 675.65 | 676.1 | 6.06 | 1 |
| 7-2 | 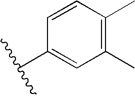 | 655.69 | 656.2 | 26.72 | 3 |
| 7-3 | 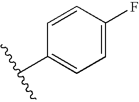 | 645.62 | 646.0 | 5.09 | 1 |
| 7-4 | 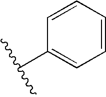 | 627.63 | 628.2 | 23.64 | 3 |

### EXAMPLE 8

### b 4-(2-{(S)-1-AMINO-3-[5-(2-FLUORO-3-METHOXYPHENYL)-3-(2-FLUORO-6-TRIFLUOROMETHYLBENZYL)-2,6-DIOXO-3,6-DIHYDRO-2H-PYRIMIDIN-1-YL]-PROPYL}-PHENOXY-BUTYRIC ACID (8-1)

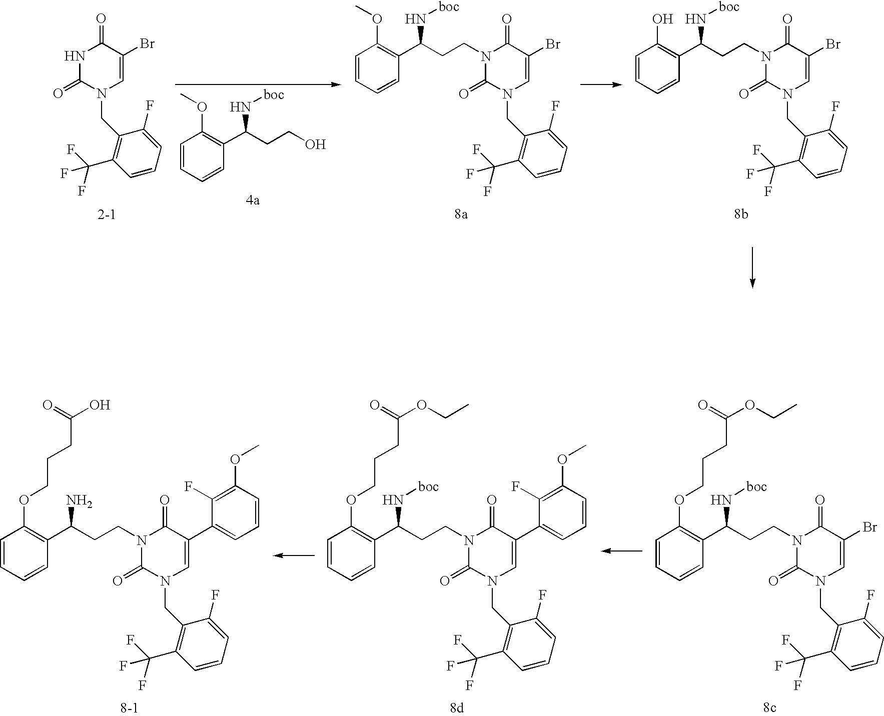

Step 8A:

Triphenylphosphine (Ph₃P, 2.45 g, 9.35 mmol) was added to the suspension of compound 2-1 (2.29 g, 6.23 mmol) in dry THF (20 mL), followed by addition of diisopropyl azadicarboxylate (DIAD, 1.84 mL, 9.35 mmol). The solution became clear quickly and was stirred at room temperature for 3 hrs. An additional 1 eq. of Ph₃P and DIAD were added. The mixture was stirred at room temperature overnight. The mixture was diluted with ethyl acetate, washed with water, brine, dried over MgSO₄ and purified by column chromatography to give 4.07 g of 8a. MS (CI) in/z 528.1/530.1 (MH⁺), HPLC: t_{R}=2.74 min (Method 2).

Step 8B:

BBr₃ (1M in dichloromethane, 13 mL, 13 mmol) was added to a solution of 8a in dichloromethane at −78° C. The mixture was stirred overnight while slowly warming to room temperature. The solvent was evaporated and MeOH was added to destroy any remaining BBr₃. After evaporation, the remaining material was then dissolved in dichloromethane/i-PrOH(3/1) and washed with saturated NaHCO₃ and brine. The organic layer was dried over MgSO₄ and then concentrated. MS (CI) m/z 516.3/518.3 (MH⁺), HPLC: t_{R}=2.54 min (Method 2).

The residue was dissolved in dichloromethane (10 mL) and TEA (0.37 mL, 2.67 mmol) was added followed by the addition of di-tert-butyldicarbonate (0.61 mL, 2.67 mmol). The mixture was stirred at room temperature over night. Dichloromethane was added and the solution was then washed with water, brine and dried over MgSO₄. Concentration and purification by column chromatography (5% acetonitrile/dichloromethane) gave 604 mg of 8b. HPLC: t_{R}=2.94 min (Method 2). MS (CI) m/z: 513.9/515.9 (MH⁺-Boc).

Step 8C:

The ethyl 4-bromobutyrate(0.2 mL, 1.36 mmol) was added to the solution of 8b (560 mg, 0.91 mmol) in DMF (3 mL), followed by addition of K₂CO₃ (251 mg, 1.82 mmol). The mixture was heated at 70° C. for 4 hours. It was cooled to room temperature, diluted with ethyl acetate. The organic layer is then washed with water, brine and dried over MgSO₄. It was then concentrated and purified by prep TLC plate (40% ethyl acetate/hexane) to yield 550 mg of 8c. HPLC: t_{R}=3.19 min (Method 2). MS (CI) m/z: 629.9/631.9 (MH⁺-Boc)

Step 8D:

To 8c (260 mg, 0.356 mmol) in a mixture of dioxane/H₂O (6/1, 7 mL), under N₂ flow, Na₂CO₃ (226 mg, 2.14 mmol), 2-fluoro-3-methoxyphenylboronic acid (121 mg, 0.71 mmol), Pd(Ph₃P)₄ (41 mg, 0.036 mmol) were added. The mixture was sealed and heated at 100° C. with stirring over night. The mixture was then extracted and washed with water, brine and dried over MgSO₄. It was then concentrated and purified by prep TLC plate (40% ethyl acetate/hexane) to give 165 mg of the desired product 8d. MS (CI) m/z: 676.1(MH⁺-Boc).

Step 8E:

To 8d (165 mg, 0.21 mmol) in THF (3 mL), LiOH (50.4 mg, 2.1 mmol) and 3 drops of water was added. The mixture was stirred at 60° C. overnight. It was diluted with ethyl acetate and acidified by 1N HCl. Organic layer was washed with brine, dried and purified by prep TLC plate (10% MeOH/dichloromethane) to yield the intermediate acid, which was treated with 50% TFA/DCM (2 mL) for 30 min. and then concentrated and purified by prep TLC plate (10% MeOH/DCM). The pure material was treated with 1 eq. of 0.1 N NaOH in water (10 mL) and lyophilized to yield 76 mg of 8-1 as white sodium salt. NMR (CDCl₃), δ, 7.53-7.42 (2 H, m), 7.33-7.27 (1 H, m), 7.19-7.11 (2 H, m), 7.00 (1 H, s), 7.02-6.76 (5 H, m), 5.15 (2 H, m), 4.25-4.15 (1 H, m), 4.08-3.92 (2 H, m), 3.90-3.54 (2 H, m), 3.79 (3 H, s), 2.35-2.05 (4 H, m), 2.00-1.75 (2 H, m). MS (CI) m/z 648.0 (MH⁺), HPLC: t_{R}=5.74 min (Method 1).

### EXAMPLE 9

### (2-{2-[3-(2-FLUORO-6-TRIFLUOROMETHYL-BENZYL)-5-(3-ISOPROPYL-PHENYL)-4-METHYL-2,6-DIOXO-3,6-DIHYDRO-2 H-PYRIMIDIN-1-YL]-1-METHYLAMINO-ETHYL}-PHENOXY)-ACETIC ACID

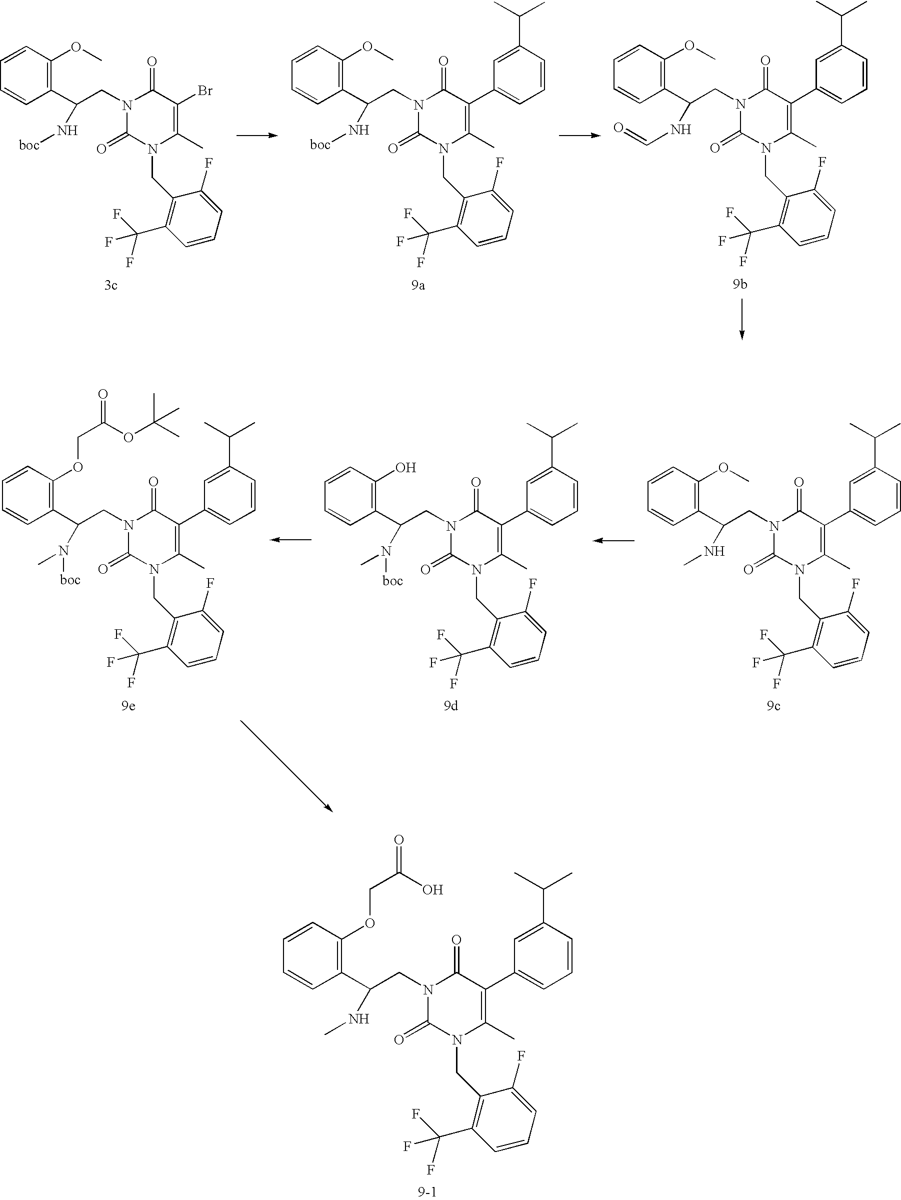

Step 9A:

Following the procedure of Step 3G, to compound 3c (5.5 g, 8.73 mmol) in a mixture of dioxane/water (9/1, 50 mL) in a sealable tube was added 3-isopropylbenzeneboronic acid (2.15 g, 13.1 mmol), Na₂CO₃ (5.55 g, 52.4 mmol) and Pd(PPh₃)₄ (1.0 g, 0.873 mmol) resulting in 4.0 g of 9a.

Step 9B:

Compound 9a (2 g, 3.0 mmol) was stirred in a mixture of trifluoroacetic acid/dichloromethane (20 mL) for 2 hours, then concentrated and redissolved in dichloromethane /isopropyl alcohol (3/1, 40 mL). The solution was washed with NaHCO₃, dried over MgSO₄, and concentrated. The residue was heated at 60° C. with ethyl formate (10 mL) for 18 hours. The mixture was concentrated and purified by silica gel chromatography to give 9b as a white solid (1.5 g). MS (CI) m/z: 598.1(MH⁺). HPLC t_{R}=3.17 min (Method 2).

Step 9C:

To 9b (1.5 g, 2.51 mmol) in dioxane (8.0 mL), was added NaBH₄ (0.48 g, 12.6 mmol), then acetic acid (0.8 mL) at 0° C. The mixture was heated at 60° C. for 2 hours and then was quenched with water. The mixture was extracted with ethyl acetate, the organic layers were combined, dried over MgSO₄ and concentrated to give 9c. MS (CI) m/z: 584.1(MH⁺). HPLC t_{R}=2.09 min (Method 2).

Step 9D:

Following the procedure of Step 5A, 9c and di-tert-butyldicarbonate yielded 9d (0.71 g). MS (CI) m/z: 670.1(MH⁺). HPLC t_{R}=2.97 min (Method 2).

Step 9E:

To 9d (0.25 g, 0.37 mmol) in DMF (3 mL), was added t-butyl bromoacetate (0.08 mL, 0.56 mmol) and K₂CO₃ (0.1 g, 0.74 mmol). The mixture was heated at 100° C. for 3 min in a microwave. The mixture was then diluted with ethyl acetate, washed with water and brine, dried over MgSO4, and concentrated. Purification by prep TLC using 40% ethyl acetate in hexane gave 9e (146 mg). MS (CI) m/z: 684.1 (MH⁺).

Step 9F:

9e was stirred in 50% trifluoroacetic acid in dichloromethane (2 mL) for 30 min. The mixture was concentrated and purified by prep-TLC plate using 10% MeOH in dichloromethane to yield 9-1. MS (CI) m/z: 628.0 (MH+). HPLC t_{R}=2.54 min (Method 2).

### EXAMPLE 10

### 3-{2-AMINO-2-[2-(2 H-TETRAZOL-5-YLMETHOXY)-PHENYL]-ETHYL}-5-(2-FLUORO-3-METHOXY-PHENYL)-1-(2-FLUORO-6-TRIFLUOROMETHYL-BENZYL)-6-METHYL-1 H-PYRIMIDINE-2,4-DIONE

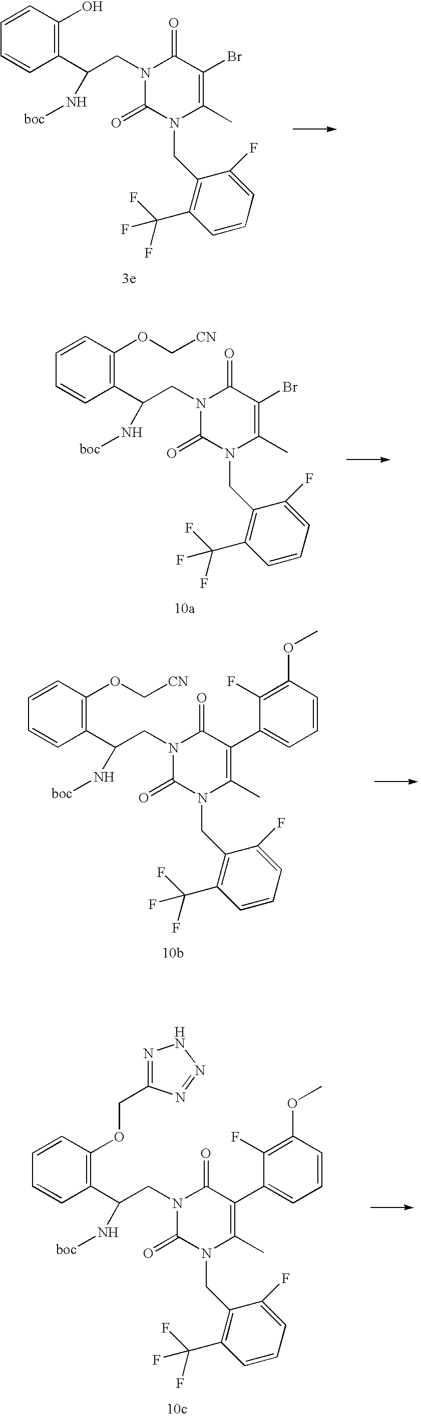

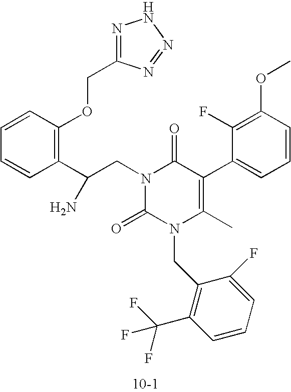

Step 10A:

Chloroacetonitrile(48 mg, 1.3 mmol) and K₂CO₃ (180 mg, 1.3 mmol) were added to 3e (400 mg, 0.65 mmol) in DMF (2 mL). The mixture was stirred at 60° C. for 16 hours and was partitioned between water and ethyl acetate. The ethyl acetate layer was separated and dried. Silica gel chromatography using 3% acetonitrile in dichloromethane gave 10a (404 mg). MS (CI) m/z: 554.9/556.9 (MH⁺-Boc). HPLC t_{R}=2.75 min (Method 2).

Step 10B:

10a (400 mg, 0.6 mmol) was converted into 10b (300 mg) using Suzuki coupling conditions as shown in Step 3G. MS (CI) m/z: 601.0 (MH⁺-Boc). HPLC t_{R}=2.78 min (Method 2).

Step 10C:

Triethylaluminum (25% in toluene, 0.234 mL, 0.435 mmol) and azidotributyltin (0.12 mL, 0.435 mmol) were added to 10b (102 mg, 0.145 mmol) in toluene (2 mL). The mixture was heated at 80° C. for 6 hours, then the mixture was partitioned between dichloromethane and water. The organic layer was washed with 1 N HCl, water, and brine and was dried over MgSO₄. Concentration followed by purification by prep-TLC plate gave 10c (40 mg). Compound 10c was treated with 50% trifluoroacetic acid in dichloromethane (1 mL) for 3 hours. The mixture was concentrated and purification by prep-TLC using 10% MeOH in dichloromethane with 1% NH₄OH as elutant gave 10-1 (14 mg). MS (CI) m/z: 644.0 (MH⁺). HPLC t_{R}=2.43 min (Method 2).

### EXAMPLE 11

### (4-{1-AMINO-2-[3-(2-FLUORO-6-TRIFLUOROMETHYL-BENZYL)-5-(3-METHOXY-PHENYL)-4-METHYL-2,6-DIOXO-3,6-DIHYDRO-2H-PYRIMIDIN-1-YL]-ETHYL}-PHENOXY)-ACETIC ACID

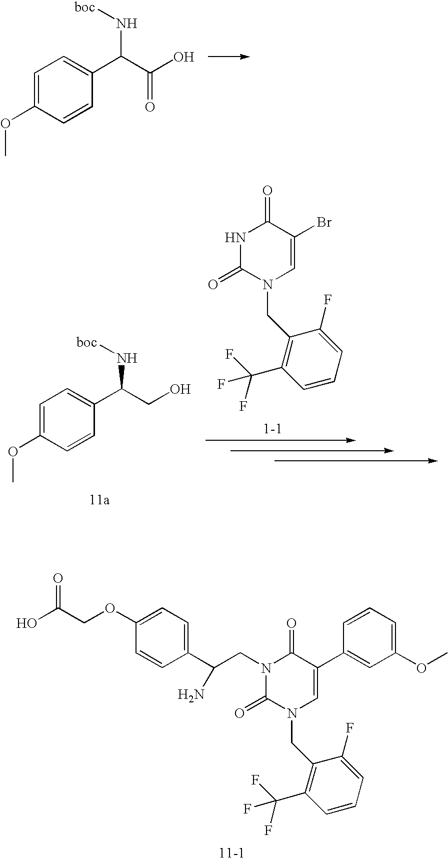

Step 11A:

Following the procedure of Step 4A, Boc-(S)-2-amino-2-(4′-methoxyphenyl)-acetic acid was reduced to the alcohol 11a. Following the procedure as shown in Steps 3C through 3G, compound 11a gave example 11-1. MS (CI) m/z: 602.0 (MH⁺). HPLC t_{R}=5.62 min (Method 1).

The following compounds were made using the above procedures:

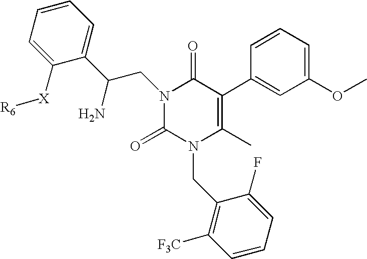

| | | | | t_{R} (min) |
|---|---|---|---|---|
| Ex | —X—R₆ | MW | MH⁺ | Method 1 |
| 11-1 | 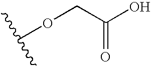 | 601.55 | 602.0 | 5.62 |
| 11-2 | 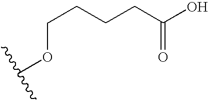 | 643.63 | 644.0 | 5.93 |
| 11-3 | 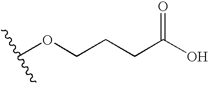 | 629.60 | 630.0 | 4.93 |

It will be appreciated that, although specific embodiments of the invention have been described herein for purposes of illustration, various modifications may be made without departing from the spirit and scope of the invention. Accordingly, the invention is not limited except as by the appended claims.

We claim:

1. A compound having the structure: 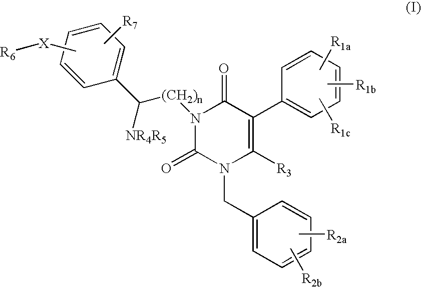
or a stereoisomer, prodrug or pharmaceutically acceptable salt, ester or solvate thereof, wherein:
R₁ₐ, R_{1b} and R_{1c}, are the same or different and independently hydrogen, halogen, C₁₋₄alkyl or alkoxy;
R₂ₐ and R_{2b} are the same or different and independently hydrogen, halogen, trifluoromethyl, cyano or —SO₂CH₃;
R₃ is hydrogen or methyl;
R₄ and R₅ are the same or different and independently hydrogen or lower alkyl;
R₆ is —COOH or an acid isostere;
R₇ is hydrogen, halogen or C₁₋₆alkyl;
n is 1 or 2; and
X is —(C₁₋₆alkanediyl)-O—, where C₁₋₆alkanediyl is optionally substituted with from 1 to 3 C₁₋₄alkyl groups.

2. The compound of claim 1 wherein R₁ₐ is halogen.

3. The compound of claim 2 wherein R₁ₐ is fluoro or chloro.

4. The compound of claim 1 wherein R_{1b} is alkoxy.

5. The compound of claim 1 wherein R_{1c} is hydrogen.

6. The compound of claim 1 wherein R₂ₐ is halogen.

7. The compound of claim 1 wherein R_{2b} is hydrogen, halogen, trifluoromethyl or —SO₂CH₃.

8. The compound of claim 1 wherein R₃ is hydrogen.

9. The compound of claim 1 wherein R₃ is methyl.

10. The compound of claim 1 wherein R₄ is hydrogen.

11. The compound of claim 1 wherein R₄ is methyl.

12. The compound of claim 1 wherein R₅ is hydrogen or methyl.

13. The compound of claim 1 wherein R₆ is —COOH.

14. The compound of claim 1 wherein R₆ is an acid isostere.

15. The compound of claim 1 wherein X is —CH₂-O—.

16. The compound of claim 1 wherein X is —CH₂CH₂-O—.

17. The compound of claim 1 wherein X is —CH₂CH₂CH₂-O—.

18. The compound of claim 1 wherein X is —CH₂CH₂CH₂CH₂-O—.

19. The compound of claim 1 wherein n is 1.

20. The compound of claim 1 wherein n is 2.

21. A pharmaceutical composition comprising a compound of claim 1 and a pharmaceutically acceptable carrier or diluent.

22. A method for antagonizing gonadotropin-releasing hormone in a subject in need thereof, comprising administering to the subject an effective amount of a compound of claim 1.

23. A method for treating a sex-hormone related condition of a subject in need thereof, comprising administering to the subject an effective amount of the pharmaceutical composition of claim 21.

24. The method of claim 23 wherein the sex-hormone related condition is cancer, benign prostatic hypertrophy or myoma of the uterus.

25. The method of claim 24 wherein the cancer is prostatic cancer, uterine cancer, breast cancer or pituitary gonadotroph adenomas.

26. The method of claim 23 wherein the sex-hormone related condition is endometriosis, polycystic ovarian disease, uterine fibroids or precocious puberty.

27. A method for preventing pregnancy of a subject in need thereof, comprising administering to the subject an effective amount of the pharmaceutical composition of claim 21.

28. A method for treating lupus erythematosis, irritable bowel syndrome, premenstrual syndrome, hirsutism, short stature or sleep disorders of a subject in need thereof, comprising administering to the subject an effective amount of the pharmaceutical composition of claim 21.
